SEQUENCE LISTING (25 sequences)

SEQ ID NO: 1 - protein, 9 aa, Unknown
  Balb/c AAV epitope
IPQYGYLTL

SEQ ID NO: 2 - protein, 4 aa, Unknown
  heparin binding site
  MISC_FEATURE (2)..(2): can be any amino acid
  MISC_FEATURE (3)..(3): can be any amino acid
RXXR

SEQ ID NO: 3 - protein, 735 aa, adeno-associated virus 2 [sequence omitted]

SEQ ID NO: 4 - protein, 736 aa, Unknown
  adeno-associated virus 6
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPFGLVEEGAKTAPGKKRPVEQSPQEPDSSSGIGKTGQQPAKKRLNFGQTGDSESVPDPQPLGEPPATPAAVGPTTMASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFTFSYTFEDVPFHSSYAHSQSLDRLMNPLIDQYLYYLNRTQNQSGSAQNKDLLFSRGSPAGMSVQPKNWLPGPCYRQQRVSKTKTDNNNSNFTWTGASKYNLNGRESIINPGTAMASHKDDKDKFFPMSGVMIFGKESAGASNTALDNVMITDEEEIKATNPVATERFGTVAVNLQSSSTDPATGDVHVMGALPGMVWQDRDVYLQGPIWAKIPHTDGHFHPSPLMGGFGLKHPPPQILIKNTPVPANPPAEFSATKFASFITQYSTGQVSVEIEWELQKENSKRWNPEVQYTSNYAKSANVDFTVDNNGLYTEPRPIGTRYLTRPL

SEQ ID NO: 5 - protein, 738 aa, Unknown
  adeno-associated virus rh64
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPVEPSPQRSPDSSTGIGKKGQQPARKRLNFGQTGDSESVPDPQPIGEPPAAPSSVGSGTMAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFSFSYTFEDVPFHSSYAHSQSLDRLMNPLIDQYLYYLSRTQSTGGTAGTQQLLFSQAGPSNMSAQARNWLPGPCYRQQRVSTTLSQNNNSNFAWTGATKYHLNGRDSLVNPGVAMATNKDDEDRFFPSSGILMFGKQGAGKDNVDYSNVMLTSEEEIKTTNPVATEQYGVVADNLQQQNTAPIVGAVNSQGALPGMVWQNRDVYLQGPIWAKIPHTDGNFHPSPLMGGFGLKHPPPQILIKNTPVPADPPTAFNQAKLNSFITQYSTGQVSVEIVWELQKENSKRRNPEIQYTSNYYKSTNVDFAVNTEGVYSEPRPIGTRYLTRNL

SEQ ID NO: 6 - protein, 736 aa, Unknown
  adeno-associated virus rh8
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPVEQSPQEPDSSSGIGKTGQQPAKKRLNFGQTGDSESVPDPQPLGEPPAAPSGLGPNTMASGGGAPMADNNEGADGVGNSSGNWHCDSTWLGDRVITTSTRTWALPTYNNHLYKQISNGTSGGSTNDNTYFGYSTPWGYFDFNRFHCHFSPRDWQRLINNNWGFRPKRLNFKLFNIQVKEVTTNEGTKTIANNLTSTVQVFTDSEYQLPYVLGSAHQGCLPPFPADVFMVPQYGYLTLNNGSQALGRSSFYCLEYFPSQMLRTGNNFQFSYTFEDVPFHSSYAHSQSLDRLMNPLIDQYLYYLVRTQTTGTGGTQTLAFSQAGPSSMANQARNWVPGPCYRQQRVSTTTNQNNNSNFAWTGAAKFKLNGRDSLMNPGVAMASHKDDDDRFFPSSGVLIFGKQGAGNDGVDYSQVLITDEEEIKATNPVATEEYGAVAINNQAANTQAQTGLVHNQGVIPGMVWQNRDVYLQGPIWAKIPHTDGNFHPSPLMGGFGLKHPPPQILIKNTPVPADPPLTFNQAKLNSFITQYSTGQVSVEIEWELQKENSKRWNPEIQYTSNYYKSTNVDFAVNTEGVYSEPRPIGTRYLTRNL

SEQ ID NO: 7 - protein, 735 aa, Unknown
  adeno-associated virus hu.51
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVGEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSGYAHSQSLDRLMNPLIDQYLYYLSTTNTPSGTTTQSRLQFSQAGASDIRDQSRNWLPGPCYRQQRVSKTSADNNNSEYSWTGATKYHLNGRDSLVNPGPAMASHKDNEEKFFPQSGVLIFGKQGSEKTNVDIEKVMITDEEEIRTTNPVATEQYGSVSTNLQRGNRQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 8 - protein, 735 aa, Unknown
  adeno-associated virus hu.34
MAADGYLPDWLEDTLSEGIRQRWKLKPGPPPPEPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNESQAVGRSSFYCLEYFPSQMLRTGNNFTFSYTFEDVPFHSSYAHSQSLGRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNRQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 9 - protein, 735 aa, Unknown
  adeno-associated virus hu.35
MAADGYLPDWLEDTLSEGIRQRWKLKPGPPPPEPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLGRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNRQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 10 - protein, 735 aa, Unknown
  adeno-associated virus hu.45
MAADGYLPDWLEDTLSEGIRQWWKLKPGPPPPKPAERHRDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSGYQLPYVLGSAHQGCLPPFPADVFMVPQYGYPTLNNGSQAVGRSSFYCLEYFPSQMLRTGNNFTFSYTFEDVPFHSSYAHSQSLDRLMNPLIDQYLYYLSTTNTPSGTTTQSRLQFSQAGASDIRDQSRNWLPGPCYRQQRVSKTSADNNNSEYSWTGATKYHLNGRDSLVNPGPAVASHKDDEEKFFPQSGVLIFGKQGSEKTNVDIEKVMITDEEEIRTTNPVATEQYGSVSTNLQRGNRQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 11 - protein, 735 aa, Unknown
  adeno-associated virus hu.47
MAADGYLPDWLEDTLSEGIRQWWKLKPGPPPPKPAERHRDDSRGLVLPGYKYLGPFNGLDKGEPVNEADAAALEHDKAYDRQLDSGDNPYLKYNHADAEFQERLKEDTSFGGNLGRAVFQAKKRVLEPLGLVGEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDSHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSTTNTPSGTTTQSRLQFSQAGASDIRDQSRNWLPGPCYRQQRVSKTSADNNNSEYSWTGATKYHLNGRDSLVNPGPAMASHKDNEEKFFPQSGVLIFGKQGSEKTNVDIEKVMITDEEEIRTTNPVATEQYGSVSTNLQRGNRQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 12 - protein, 735 aa, Unknown
  adeno-associated virus hu.13 capsid
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPAEPDSSSGTGKAGQQPARKRLNFGQTGDADSVPDPQPLGQPPAAPSGLGTNTMASGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQGGNTQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 13 - protein, 738 aa, Human adenovirus type 8
MAADGYLPDWLEDNLSEGIREWWALKPGAPKPKANQQKQDDGRGLVLPGYKYLGPFNGLDKGEPVNAADAAALEHDKAYDQQLQAGDNPYLRYNHADAEFQERLQEDTSFGGNLGRAVFQAKKRVLEPLGLVEEGAKTAPGKKRPVEPSPQRSPDSSTGIGKKGQQPARKRLNFGQTGDSESVPDPQPLGEPPAAPSGVGPNTMAAGGGAPMADNNEGADGVGSSSGNWHCDSTWLGDRVITTSTRTWALPTYNNHLYKQISNGTSGGATNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFQFTYTFEDVPFHSSYAHSQSLDRLMNPLIDQYLYYLSRTQTTGGTANTQTLGFSQGGPNTMANQAKNWLPGPCYRQQRVSTTTGQNNNSNFAWTAGTKYHLNGRNSLANPGIAMATHKDDEERFFPSNGILIFGKQNAARDNADYSDVMLTSEEEIKTTNPVATEEYGIVADNLQQQNTAPQIGTVNSQGALPGMVWQNRDVYLQGPIWAKIPHTDGNFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPRPIGTRYLTRNL

SEQ ID NO: 14 - protein, 737 aa, Human adenovirus type 7
MAADGYLPDWLEDNLSEGIREWWDLKPGAPKPKANQQKQDNGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPAKKRPVEPSPQRSPDSSTGIGKKGQQPARKRLNFGQTGDSESVPDPQPLGEPPAAPSSVGSGTVAAGGGAPMADNNEGADGVGNASGNWHCDSTWLGDRVITTSTRTWALPTYNNHLYKQISSETAGSTNDNTYFGYSTPWGYFDFNRFHCHFSPRDWQRLINNNWGFRPKKLRFKLFNIQVKEVTTNDGVTTIANNLTSTIQVFSDSEYQLPYVLGSAHQGCLPPFPADVFMIPQYGYLTLNNGSQSVGRSSFYCLEYFPSQMLRTGNNFEFSYSFEDVPFHSSYAHSQSLDRLMNPLIDQYLYYLARTQSNPGGTAGNRELQFYQGGPSTMAEQAKNWLPGPCFRQQRVSKTLDQNNNSNFAWTGATKYHLNGRNSLVNPGVAMATHKDDEDRFFPSSGVLIFGKTGATNKTTLENVLMTNEEEIRPTNPVATEEYGIVSSNLQAANTAAQTQVVNNQGALPGMVWQNRDVYLQGPIWAKIPHTDGNFHPSPLMGGFGLKHPPPQILIKNTPVPANPPEVFTPAKFASFITQYSTGQVSVEIEWELQKENSKRWNPEIQYTSNFEKQTGVDFAVDSQGVYSEPRPIGTRYLTRNL

SEQ ID NO: 15 - protein, 735 aa, Unknown
  adeno-associated virus hu.29R
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPAEPDSSSGTGKSGNQPARKRLNFGQTGDSDSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLGYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGPEKTNVDIEKVMITDEEEIRTTNPVATEQYGSVSTNLQSGNTQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 16 - protein, 4 aa, Unknown
  heparin binding site
RGNR

SEQ ID NO: 17 - protein, 4 aa, Unknown
  heparin binding site
RQNR

SEQ ID NO: 18 - protein, 4 aa, Unknown
  heparin binding site
SGNT

SEQ ID NO: 19 - protein, 4 aa, Unknown
  heparin binding site
GGNT

SEQ ID NO: 20 - protein, 4 aa, Unknown
  heparin binding site
QQNT

SEQ ID NO: 21 - protein, 4 aa, Unknown
  heparin binding site
AANT

SEQ ID NO: 22 - protein, 9 aa, Unknown
  epitope
VPQYGYLTL

SEQ ID NO: 23 - protein, 9 aa, Unknown
  epitope
TSNYNKSVN

SEQ ID NO: 24 - protein, 9 aa, Unknown
  epitope
NSLVNPGVA

SEQ ID NO: 25 - protein, 9 aa, Unknown
  epitope
NSLANPGIA

(19) United States
(12) Patent Application Publication
(10) Pub. No.: US 2009/0317417 A1
(43) Pub. Date: Dec. 24, 2009

(54) Modified AAV Vectors Having Reduced Capsid Immunogenicity and Use Thereof

(75) Inventors: Luc H. Vandenberghe, Philadelphia, PA (US); James M. Wilson, Gladwyne, PA (US)

Correspondence Address: HOWSON & HOWSON LLP, 501 OFFICE CENTER DRIVE, SUITE 210, FORT WASHINGTON, PA, 19034, US

(73) Assignee: The Trustees of the University of Pennsylvania, Philadelphia, PA (US)

(21) Appl. No.: 12/226,536

(22) PCT Filed: Apr. 27, 2007
(86) PCT No.: PCT/US2007/010056

§ 371 Date: Oct. 21, 2008

Related U.S. Application Data
(60) Provisional application No. 60/795,965, filed on Apr. 28, 2006.

(51) Int. Cl. A61K 39/235; C12N 7/00; A61P 31/12

(52) U.S. Cl. 424/186.1; 435/235.1

(57) ABSTRACT

A method of reducing the cellular immune response and/or toxicity of AAV-mediated delivery is described. The method provides for masking or ablating a RxxR motif which induces T-cells, and which is located on select AAV capsids. The method further provides for reducing or eliminating heparin binding to an AAV. Also provided are compositions containing modified AAV capsids and methods of using same.

### STATEMENT REGARDING FEDERALLY SPONSORED RESEARCH OR DEVELOPMENT

This application describes work supported at least in part by a grant from the National Institutes of Health, NHLBI grant number P01-HL-059407. The US government may have certain rights in this invention.

### BACKGROUND OF THE INVENTION

The present invention provides methods of altering the immunogenicity of an AAV.

Adeno-associated virus (AAV), a member of the Parvovirus family, is a small nonenveloped, icosahedral virus with a single-stranded linear DNA genome of 4.7 kilobases (kb) to 6 kb. AAV is assigned to the genus, Dependovirus, because the virus was discovered as a contaminant in purified adenovirus stocks. AAV's life cycle includes a latent phase at which AAV genomes, after infection, are integrated into host genomes and an infectious phase in which, following either adenovirus or herpes simplex virus infection, the integrated AAV genomes are subsequently rescued, replicated, and packaged into infectious viruses. The properties of non-pathogenicity, broad host range of infectivity, including non-dividing cells, and integration make AAV an attractive delivery vehicle.

A variety of different AAV sequences and methods for isolating same from tissues have been described. AAV1-6, AAV7, AAV9 and AAV9, amongst other AAV sequences obtained from simian or human tissue sources have been described. See, e.g., International Patent Publication Nos. WO 02/33269, WO 02/386122 (AAV8), and International Patent Publication No. WO 2005/033321. With this, a move away from defining AAV strictly by serologic cross-reactivity (serotypes) has occurred. Recent literature defines the relationship between these AAV in terms of phylogenetic relatedness, proposing groups termed “clades”. See, e.g., Gao et al, J Virol, 78(12):6381-6388 (June 2004); International Patent Publication No. WO 2005/033321.

AAV is currently being considered as a delivery vector for gene therapy in the clinic. Activation of T cells to the capsid of adeno-associated virus (AAV) serotype 2 vectors has been implicated in liver toxicity in a recent human gene therapy trial of hemophilia B. [Manno, C. S. et al. Successful transduction of liver in hemophilia by AAV-Factor IX and limitations imposed by the host immune response. Nat Med (2006)].

AAV2 is known to bind heparin and heparan sulfate proteoglycan (HSPG) on the cell surface. The 585RGNR588 motif on the capsid of the virion has been mapped as the domain responsible for this interaction. A. Kern, et al, J Virol 77:11072-81 (2003); S. R. Opie et al., J Virol 77:6995-7006 (2003). Further, an attempt to improve altering the tropism of AAV by delivering a non-HSPG-binding AAV2 in vivo has been described.

What are needed are AAV compositions having altered immune effects.

### SUMMARY OF THE INVENTION

In one aspect, the present invention provides compositions in which the functionality of a heparin binding domain is altered in AAV in order to reduce the immunogenicity, and particularly, T-cell responses directed to the AAV. In one embodiment, the invention provides compositions in which the heparin binding domain of a selected AAV is masked or ablated, to increase safety and success of the gene delivery.

In another aspect, the invention provides pharmaceutical and vaccine compositions containing the modified AAV of the invention and a physiologically compatible carrier are provided.

In still another aspect, methods of delivering the pharmaceutical and vaccine compositions of the invention are described.

Still other advantages of the present invention will be apparent from the detailed description of the invention.

### BRIEF DESCRIPTION OF THE DRAWINGS

FIG. 1A is a bar graph that shows the number of IFN-γ Spot Forming Units (SFU) in an ELISPOT assay of splenocytes harvested 7 days after intra-muscular injection of AAV in C57Bl/6 mice. Several Clade B naturally occurring AAV isolates (AAV2, hu.51, hu.29R, hu.13) and mutants (AAV2HSPG-) were evaluated for T cell responses. In addition, mice were injected with AAV 2/7 (denoted as AAV7), AAV2/8 (denoted as AAV8) and the AAV2/8 mutant RQNR (denoted as AAV8RZNR) to monitor T cell activation. All AAVs are identified along the x axis of FIG. 1B. Cells were stimulated with 3 peptide pools (identified as A, B and C) together spanning the entire AAV2 or AAV8 capsid, the C57Bl/6 dominant epitope for AAV2 or AAV8 (solid bar) or a no peptide negative control (open bar).

FIG. 1B is a bar graph that shows the number of IFN-γ Spot Forming Units (SFU) in an ELISPOT assay of splenocytes harvested 14 days after intra-muscular injection of AAV in Balb/C mice. AAV2, AAV2/hu.51 and the AAV2HSPG-mutant IM injected groups were stimulated with no peptide, comprehensive AAV2 peptide pools (A, B, C) and the AAV2 dominant epitope. Cells from AAV7, AAV8 and AAV8RQNR injected groups were incubated in the absence of peptide, with pooled AAV2/8 peptides or its dominant epitope peptide. In each case, the number of spots (y axis) is presented as a function of the injected vector (x axis) for different peptides (dominant and the pools) used to stimulate the cells. The key for the stimulating peptides is the same as that for FIG. 1A.

FIGS. 2A-2C are bar graphs that show the time course of T cell response to AAV capsid in cynomolgus macaques following intramuscular vaccinations with AAV vectors of different serotypes in individual monkeys. Monkeys were immunized by IM injection of a mixture of 10¹² GC each of AAV.CMV.HIVgp140, AAV.CMV.HIVGN2, and AAV.CMV.HIV RT3. At week 2, 4, 8, 14, 24 and 32 after immunization, PBMCs were isolated and stimulated in vitro with peptide pools corresponding to the specific AAV serotypes and analyzed using the INF-γ ELISPOT assay. A total of 15 animals were dosed with 5 animals per vector serotype (AAV2: FIG. 2A, AAV2/7: FIG. 2B, AAV2/8: FIG. 2C). The frequency of spots as measured by ELISPOT is presented as a function of time, noted in weeks (e.g., 8 w) for the individual animals that are identified by five digit numbers. For each assay, three peptide pools spanning the entire VP1 region of the corresponding capsids are used. (n/a: not assayed). The key identifies the stimulating peptides represented by the variously shaded bars of the graph.

FIG. 3 shows the impact of heparan sulfate proteoglycan affinity on AAV binding. The relative binding of AAVs to human monocyte derived dendritic cells (DC, bar bar), HeLa (white bar) and CHO (shaded bar) cells is compared to AAV2. Cells were incubated for 3 h at 4° C. with AAV2, AAV2HSPG-, AAV2 in the presence of heparin, AAV8 and AAV8RQNR. Cell pellets were harvested, washed 3 times with culture medium and resuspended in 400 mM NaCl solution. DNase resistant genome copies were measured by quantitative PCR and normalized with values from the AAV2-bound virus condition. Binding data are presented for each vector relative to AAV2 as fold increase; a positive number represents binding greater than AAV2 and a negative number represents binding less than AAV2.

FIG. 4 is a bar chart showing the results of immunization with a variety of AAV on T-cell activation. Balb/c mice were immunized with 1×10¹¹ GC AAV2/6, AAV2/6.1, AAV2/6.2, AAV2/6.1.2, AAV2/1 and AAV2 vector. 13 days later splenocytes were harvested from 3 mice per group and pooled. Equal amounts of splenocytes were stimulated in vitro with the Balb/c AAV epitope IPQYGYLTL [SEQ ID NO: 1] in a ELISPOT assay.

### DETAILED DESCRIPTION OF THE INVENTION

In one aspect, the invention provides a method for reducing a cellular immune response to an adeno-associated virus (AAV) delivery vehicle. In another aspect, the invention provides a method for reducing the toxicity of an AAV-delivery vehicle. In still another aspect, the invention provides compositions comprising modified AAV.

In one embodiment, the invention provides an AAV modified by preventing binding of heparin to an AAV capsid having a heparin binding site. In another embodiment, the invention provides an AAV having a capsid modified to destroy the heparin binding domain and/or T-cell activating functionality of the heparin binding domain.

In one aspect, the invention provides a composition for AAV-mediated delivery of a molecule with reduced T-cell immunogenicity. In one embodiment, such a composition contains an AAV having a modified capsid, wherein said AAV capsid comprises an AAV capsid modified to ablate a heparin binding site in the AAV capsid protein, and a physiologically compatible carrier. In one embodiment, the modified AAV is not derived from AAV2.

In one embodiment, the AAV sequence is modified to ablate the heparin binding site. By ablating the heparin binding site, is meant that the site no longer activates T cells and/or the lacks the ability to bind to heparin.

Without wishing to be bound by theory, the inventors believe that they have found a direct link between the heparin binding domain in AAV capsids and the activation of T cells. Preliminary data indicates that T cells so activated include those that produce IFN gamma (γ) in response to stimulation with peptides from the AAV capsid. Further phenotyping of those T-cell population detected CD8+ capsid specific T cells. Thus, the inventors have found that by functionally ablating heparin binding in AAV, T cell activation is reduced or eliminated.

A heparin binding domain having an Arg-Xaa-Xaa-Arg, SEQ ID NO: 2 (the RxxR motif) has been described in AAV2 (i.e., coordinates 585-588 on AAV2VP1, SEQ ID NO: 3, Kern, et al., J Virol 77:11072-81; Opie et al., J Virol 77:6995-7006, WO 02/33269, based upon the numbering system illustrated therein). Other currently described AAV lack the RxxR binding site, but bind heparin, e.g., AAV3. Thus, these other AAV have different motifs responsible for heparin affinity.

The presence of heparin binding in a selected AAV capsid can be readily identified using a variety of assay formats (e.g., a heparin binding column), many of which use heparin or portions thereof. Once binding is identified is a selected AAV capsid, the heparin binding domain can be mapped using techniques known to those of skill in the art. For example, AAV6 sequence has been found have a heparin binding domain, which is ablated by a non-conservative amino acid change of the lysine residue at position 531. [The sequence of AAV6.1 is provided in International Patent Appln No. PCT/US06/13375 and the residue number is based on the numbering scheme provided in that international application (see, e.g., Table)]. Once the binding site of an AAV particle is mapped, as is the case for AAV2, the absence or presence of such a site can be easily determined by making use of alignment software. Homology of the binding domain predicts functional heparin binding and the absence of homology predicts the lack thereof. The functional activity, i.e., heparin affinity of the viral vectors that contain the binding site can be readily determined using known methods, including, e.g., use of a heparin binding assays [Opie, S. R., et al., J Virol 77, 6995-7006 (2003)].

Analogous regions of other AAV can be readily determined by performing an alignment of a selected AAV and AAV2 using available computer programs and well-know techniques. “Aligned” sequences or “alignments” refer to multiple nucleic acid sequences or protein (amino acids) sequences, often containing corrections for missing or additional bases or amino acids as compared to a reference sequence. The reference sequence may be AAV2, or another selected sequence. See, e.g., AAV 1 (U.S. Pat. No. 6,759,237), AAV2, AAV3, AAV4, AAV5, AAV6, AAV7, AAV8, AAV9, rh32.33, rh.10, hu.11, others AAV from human and non-human sources, see, e.g., International Patent Publication Nos. WO 02/33269, WO 02/386122 (AAV8), and GenBank, and such sequences as have been altered to correct singleton errors, e.g., AAV6.2, [AAV6, SEQ ID NO: 4, with F129L], AAV6.1 [AAV6, SEQ ID NO: 4, with a K531E change], AAV6.1.2 [AAV6, SEQ ID NO: 4, with K531E,F129L], rh.32.33, rh.10, and rh64R1 [SEQ ID NO: 5, with a R697W] and rh8R [SEQ ID NO: 6, with D531E] [see, e.g., WO 2006/110689, published Oct. 19, 2006]. Alternatively, other AAV sequences including those identified by one of skill in the art using known techniques [See, e.g., International Patent Publication No. WO 2005/033321 and GenBank] or by other means may be modified as described herein.

Alignments are performed using any of a variety of publicly or commercially available Multiple Sequence Alignment Programs. Examples of such programs include, “Clustal W”, “CAP Sequence Assembly”, “MAP”, and “MEME”, which are accessible through Web Servers on the internet. Other sources for such programs are known to those of skill in the art. Alternatively, Vector NTI utilities are also used. There are also a number of algorithms known in the art that can be used to measure nucleotide sequence identity, including those contained in the programs described above. As another example, polynucleotide sequences can be compared using Fasta™, a program in GCG Version 6.1. Fasta™ provides alignments and percent sequence identity of the regions of the best overlap between the query and search sequences. For instance, percent sequence identity between nucleic acid sequences can be determined using the Fasta™ program with its default parameters (a word size of 6 and the NOPAM factor for the scoring matrix) as provided in GCG Version 6.1, herein incorporated by reference. Multiple sequence alignment programs are also available for amino acid sequences, e.g., the “Clustal X”, “MAP”, “PIMA”, “MSA”, “BLOCKMAKER”, “MEME”, and “Match-Box” programs. Generally, any of these programs are used at default settings, although one of skill in the art can alter these settings as needed. Alternatively, one of skill in the art can utilize another algorithm or computer program which provides at least the level of identity or alignment as that provided by the referenced algorithms and programs. See, e.g., J. D. Thomson et al, Nucl. Acids. Res., “A comprehensive comparison of multiple sequence alignments”, 27(13):2682-2690 (1999).

Certain AAV sequences are natively devoid of such a heparin binding site. For AAV lacking a heparin binding site, e.g., AAV8, no modification of the AAV sequence, cell or media is required. The ability of an AAV capsid to bind heparin can be readily identified using a variety of assay formats and heparin or portions thereof for binding an AAV. Further, the ability of heparin to block the infectious/transduction ability of an AAV can readily be determined by one of skill in the art. A suitable assay for determining the ability of heparin to block any infection/transduction of transduction of an AAV has been described, e.g., in C. Halbert et al, J Virol, 75(14):6615-6624 (July 2001) and C. E. Walsh and H. Chao, Haemophilia, 8 (Suppl. 2), p. 60-67 (2002).

Other AAV sequences, e.g., AAV6, have a heparin binding site, but the ability of AAV6 to infect is partially inhibited, not blocked, by the presence of heparin. In another example, an AAV6 vp1 capsid sequence has been described as having a single amino acid residue that mediates heparin binding, the native lysine reside at position 531 [SEQ ID NO: 4]. [The sequence of AAV6 is provided in International Patent Appln No. PCT/US06/13375 and the residue number is based on the numbering scheme provided in that international application.

In one embodiment, the inventors have found that an AAV having a heparin binding domain and which is characterized by having any detectable amount of infectious/transduction ability blocked by heparin, do not secrete. Examples of such an AAV is AAV2, which is mostly cell associated during production, and AAV3. In one embodiment, a heparin binding domain is an Arg-Xaa-Xaa-Arg (RxxR) [SEQ ID 2] motif as has been described in AAV2 (i.e., about amino acids 585 to 588 of the AAV2 vp1 capsid protein, SEQ ID NO: 3, Kern, et al., J Virol 77:11072-81; Opie, et al., J Virol 77:6995-7006 (based upon the numbering illustrated in WO 02/33269)]. Xaa represents any amino acid. The inventors are the first to describe other AAV capsids having RxxR motifs, several of which are Clade B AAVs. Examples of such AAV capsids having RxxR motifs include, hu.51 [SEQ ID NO: 7], hu.34 [SEQ ID NO: 8], hu.35 [SEQ ID NO: 9], hu.45 [SEQ ID NO: 10], and hu.47 [SEQ ID NO: 11]. Other AAV having an RxxR domain can be readily identified by one of skill in the art from among those AAV sequences which have been described. In addition, other heparin binding sites can be readily identified in AAV using techniques known to those of skill in the art. In another example, AAV3 binds heparin; however, it does not contain the RxxR domain.

The inventors have found that by changing altering the amino acid residue(s) which forms a critical part of the heparin binding domain (motif) to contain a non-conservative amino acid change, not only is heparin binding ablated, but also, T cell activation is significantly reduced. In one embodiment, a single non-conservative amino acid change of an amino acid residue which mediates heparin binding is sufficient to ablate the function of this motif. As illustrated herein, a non-conservative change in K531 of AAV6 ablates heparin binding and T cell activation. Additionally, a single non-conservative change in either the first arginine or last arginine in an RxxR heparin binding domain will ablate heparin binding. As illustrated herein, in one embodiment, the first amino acid of the modified heparin sulfate glycoprotein binding site can be changed from Arg to, e.g., Ser or Glu. In another embodiment, the last amino acid of the modified heparin sulfate glycoprotein binding site is changed from Arg to Thr. Other suitable non-conservative amino acid changes will be apparent to those of skill in the art.

In one embodiment, the nucleic acid sequence encoding the AAV capsid heparin binding site is modified using site-specific mutagenesis techniques, in which the codon for the initial arginine and/or the last arginine of the motif is altered to make a non-conservative change in one (or both) of the amino acids. Examples of non-conservative amino acid changes include those, e.g., substitution of one amino acid with another amino acid of different chemical structure (properties), which affect protein function. The following table illustrates the most common amino acids and their properties.

| | | Hydro- | | | Aromatic or | |
|---|---|---|---|---|---|---|
| Amino acid | Abbrev. | phobic | Polar | Charged | Aliphatic | Codon |
| Alanine | Ala, A | X | — | — | — | GCU, GCC, GCA, |
| | | | | | | GCG |
| Cysteine | Cys, C | X | — | — | — | UGU, UGC |
| Aspartate | Asp, D | — | X | negative | — | GAU, GAC |
| Glutamate | Glu, E | — | X | negative | — | GAA, GAG |
| Phenylalanine | Phe, F | X | — | — | Aromatic | UUU, UUC |
| Glycine | Gly, G | X | — | — | — | GGU, GGC, GGA, |
| | | | | | | GGG |
| Histidine | His, H | — | X | positive | Aromatic | CAU, CAC |
| Isoleucine | Ile, I | X | — | — | Aliphatic | AUU, AUC, AUA |
| Lysine | Lys, K | — | X | positive | — | AAA, AAG |
| Leucine | Leu, L | X | — | — | Aliphatic | UUA, UUG, CUU, |
| | | | | | | CUC, CUA, CUG |
| Methionine | Met, M | X | — | — | — | AUG |
| Asparagine | Asn, N | — | X | — | — | AAU, AAC |
| Proline | Pro, P | X | — | — | — | CCU, CCC, CCA, |
| | | | | | | CCG |
| Glutamine | Gln, Q | — | X | — | — | CAA, CAG |
| Arginine | Arg, R | — | X | positive | — | CGU, CGC, CGA, |
| | | | | | | CGG, AGA, AGG |
| Serine | Ser, S | — | X | — | — | UCU, UCC, UCA, |
| | | | | | | UCG, AGU, AGC |
| Threonine | Thr, T | X | X | — | — | ACU, ACC, ACA, |
| | | | | | | ACG |
| Valine | Val, V | X | — | — | Aliphatic | GUU, GUC, GUA, |
| | | | | | | GUG |
| Tryptophan | Trp, W | X | — | — | Aromatic | UGG |
| Tyrosine | Tyr, Y | X | X | — | Aromatic | UAU, UAC |

Other suitable techniques for altering the coding sequence for the amino acid may be utilized: See, e.g., Sambrook et al, Molecular Cloning: A Laboratory Manual, Cold Spring Harbor Press (Cold Spring Harbor, N.Y.). In yet another embodiment, the heparin binding domain may be ablated by inserting one or more exogenous amino acid sequences in the RxxR motif, thereby destroying the motif.

In another embodiment, binding of heparin to an AAV containing a heparin binding site is ablated by methods other than altering the sequence of the heparin binding site. For example, one may provide the AAV capsid with a molecule which effectively masks the heparin binding site in the producer cell. For example, methods of providing a polyethylene glycol molecule to the viral particle have been described.

In yet another embodiment, one may modify of the target cell to eliminate or substantially reduce heparin binding, e.g., providing the cell with a heparin molecule on the cell surface (heparan sulfate proteoglycan) to the cell, either transiently or permanently. For example, one suitable techniques may involve enzymatic digestion of heparin, e.g., by enzymes such as heparinases. In another embodiment, soluble heparin can be delivered in conjunction with an AAV.

In another embodiment, the invention provides AAV capsids modified to ablate the heparin binding motif. In one embodiment, the source of the AAV capsid is an AAV other than AAV2. In another embodiment, the AAV comprises at least one modified AAV2 capsid protein [SEQ ID NO: 3], with the exception that the modifications are other than R585S and R588T of AAV2).

Production of rAAV with Novel AAV Capsids

The invention encompasses novel, modified, AAV capsids and the sequences encoding same, which are free of DNA and/or cellular material with these viruses are associated in nature. In another aspect, the present invention provides molecules that utilize the novel AAV nucleic acid and protein sequences of the invention, including fragments thereof, for production of molecules useful in delivery of a heterologous gene or other nucleic acid sequences to a target cell. The molecules of the invention which contain AAV sequences include any genetic element (vector) which may be delivered to a host cell, e.g., naked DNA, a plasmid, phage, transposon, cosmid, episome, a protein in a non-viral delivery vehicle (e.g., a lipid-based carrier), virus, etc., which transfers the sequences carried thereon. The selected vector may be delivered by any suitable method, including transfection, electroporation, liposome delivery, membrane fusion techniques, high velocity DNA-coated pellets, viral infection and protoplast fusion. The methods used to construct any embodiment of this invention are known to those with skill in nucleic acid manipulation and include genetic engineering, recombinant engineering, and synthetic techniques. See, e.g., Sambrook et al, Molecular Cloning: A Laboratory Manual, Cold Spring Harbor Press, Cold Spring Harbor, N.Y.

Suitably, a modified AAV capsid according to the present invention is utilized in the production of an infectious AAV particle, in which an expression cassette for delivery to a target cell is packaged into the modified AAV capsid.

The expression cassette, rep sequences, cap sequences, and helper functions required for producing AAV may be delivered to the packaging host cell in the form of any genetic element which transfer the sequences carried thereon. The selected genetic element may be delivered by any suitable method, including those described herein. The methods used to construct any embodiment of this invention are known to those with skill in nucleic acid manipulation and include genetic engineering, recombinant engineering, and synthetic techniques. See, e.g., Sambrook et al, Molecular Cloning: A Laboratory Manual, Cold Spring Harbor Press, Cold Spring Harbor, N.Y. Similarly, methods of generating rAAV virions are well known and the selection of a suitable method is not a limitation on the present invention. See, e.g., K. Fisher et al, J. Virol., 70:520-532 (1993) and U.S. Pat. No. 5,478,745.

Unless otherwise specified, the AAV ITRs, and other selected AAV components described herein, may be readily selected from among any AAV, including, without limitation, AAV1, AAV2, AAV3, AAV4, AAV5, AAV6, AAV7, AAV9, among others. These ITRs or other AAV components may be readily isolated using techniques available to those of skill in the art from an AAV sequence. Such AAV may be isolated or obtained from academic, commercial, or public sources (e.g., the American Type Culture Collection, Manassas, Va.). Alternatively, the AAV sequences may be obtained through synthetic or other suitable means by reference to published sequences such as are available in the literature or in databases such as, e.g., GenBank®, PubMed®, or the like.

A. The Expression Cassette

The expression cassette is composed of, at a minimum, 5′ AAV inverted terminal repeats, a nucleic acid molecule comprising a nucleic acid sequence which optionally encodes a desired product or is itself useful, operably linked to regulatory sequences which direct transcription, translation and/or expression thereof, and 3′ AAV ITRs. In one desirable embodiment, the ITRs of AAV serotype 2 are used. However, ITRs from other suitable sources may be selected. It is this minigene that is packaged into a capsid protein and delivered to a selected host cell.

1. The Nucleic Acid Sequences

In one embodiment, the nucleic acid sequences are heterologous to the AAV ITRs and are therapeutically useful. An example of a suitable sequence is, e.g, an RNA. Desirable RNA molecules include tRNA, dsRNA, ribosomal RNA, catalytic RNAs, siRNA, small hairpin RNA, trans-splicing RNA, and antisense RNAs. One example of a useful RNA sequence is a sequence which inhibits or extinguishes expression of a targeted nucleic acid sequence in the treated animal. Typically, suitable target sequences include oncologic targets and viral diseases. See, for examples of such targets the oncologic targets and viruses identified below in the section relating to immunogens.

In another embodiment, the nucleic acid sequence is heterologous to the AAV ITRs, which encodes a polypeptide, protein, or other product, of interest. The nucleic acid coding sequence is operatively linked to regulatory components in a manner which permits transgene transcription, translation, and/or expression in a host cell.

The composition of the nucleic acid sequence will depend upon the use to which the resulting vector will be put. For example, one type of sequence includes a reporter sequence, which upon expression produces a detectable signal. However, desirably, the sequence is a non-marker sequence encoding a product which is useful in biology and medicine, such as proteins, peptides, RNA, enzymes, dominant negative mutants, or catalytic RNAs.

The nucleic acid sequence may encode a single product. The invention further includes using multiple genes. In certain situations, a different gene may be used to encode each subunit of a protein, or to encode different peptides or proteins. This is desirable when the size of the DNA encoding the protein subunit is large, e.g., for an immunoglobulin, the platelet-derived growth factor, or a dystrophin protein. In order for the cell to produce the multi-subunit protein, a cell is infected with the recombinant virus containing each of the different subunits. Alternatively, different subunits of a protein may be encoded by the same transgene. In this case, a single gene includes the DNA encoding each of the subunits, with the DNA for each subunit separated by an internal ribozyme entry site (IRES). This is desirable when the size of the DNA encoding each of the subunits is small, e.g., the total size of the DNA encoding the subunits and the IRES is less than five kilobases. As an alternative to an IRES, the DNA may be separated by sequences encoding a 2A peptide, which self-cleaves in a post-translational event. See, e.g., M. L. Donnelly, et al, J. Gen. Virol., 78(Pt 1):13-21 (January 1997); Furler, S., et al, Gene Ther., 8(11):864-873 (June 2001); Klump H., et al., Gene Ther., 8(10):811-817 (May 2001). This 2A peptide is significantly smaller than an IRES, making it well suited for use when space is a limiting factor. More often, when the gene is large, consists of multi-subunits, or two genes are co-delivered, rAAV carrying the desired gene(s) or subunits are co-administered to allow them to concatamerize in vivo to form a single vector genome. In such an embodiment, a first AAV may carry an expression cassette which expresses a single gene and a second AAV may carry an expression cassette which expresses a different gene for co-expression in the host cell. However, the selected gene may encode any biologically active product or other product, e.g., a product desirable for study.

Suitable genes may be readily selected by one of skill in the art. The selection of the gene is not considered to be a limitation of this invention.

2. Regulatory Elements

In addition to the major elements identified above for the expression cassette, the vector also includes conventional control elements which are operably linked to the nucleic acid coding sequence in a manner which permits its transcription, translation and/or expression in a cell transfected with the plasmid vector or infected with the virus produced by the invention. As used herein, “operably linked” sequences include both expression control sequences that are contiguous with the gene of interest and expression control sequences that act in trans or at a distance to control the gene of interest.

Expression control sequences include appropriate transcription initiation, termination, promoter and enhancer sequences; introns, efficient RNA processing signals such as splicing and polyadenylation (polyA) signals; sequences that stabilize cytoplasmic mRNA; sequences that enhance translation efficiency (i.e., Kozak consensus sequence); sequences that enhance protein stability; and when desired, sequences that enhance secretion of the encoded product. A great number of expression control sequences, including promoters which are native, constitutive, regulatable and/or tissue-specific, are known in the art and may be utilized.

Examples of constitutive promoters include, without limitation, the retroviral Rous sarcoma virus (RSV) LTR promoter (optionally with the RSV enhancer), the cytomegalovirus (CMV) promoter (optionally with the CMV enhancer) [see, e.g., Boshart et al, Cell, 41:521-530 (1985)], the SV40 promoter, the dihydrofolate reductase promoter, the β-actin promoter, the phosphoglycerol kinase (PGK) promoter, and the EF1 promoter [Invitrogen]. Regulatable promoters allow regulation of gene expression and can be regulated by exogenously supplied compounds, environmental factors such as temperature, or the presence of a specific physiological state, e.g., acute phase, a particular differentiation state of the cell, or in replicating cells only. Regulatable promoters and regulatable systems are available from a variety of commercial sources, including, without limitation, Invitrogen, Clontech and Ariad. Many other systems have been described and can be readily selected by one of skill in the art. Examples of regulatable promoters regulated by exogenously supplied compounds, include, the zinc-inducible sheep metallothionine (MT) promoter, the dexamethasone (Dex)-inducible mouse mammary tumor virus (MMTV) promoter, the T7 polymerase promoter system [International Patent Publication No. WO 98/10088]; the ecdysone insect promoter [No et al, Proc. Natl. Acad. Sci. USA, 93:3346-3351 (1996)], the tetracycline-repressible system [Gossen et al, Proc. Natl. Acad. Sci. USA, 89:5547-5551 (1992)], the tetracycline-inducible system [Gossen et al, Science, 268:1766-1769 (1995), see also Harvey et al, Curr. Opin. Chem. Biol., 2:512-518 (1998)], the RU486-inducible system [Wang et al, Nat. Biotech., 15:239-243 (1997) and Wang et al, Gene Ther., 4:432-441 (1997)] and the rapamycin-inducible system [Magari et al, J. Clin. Invest., 100:2865-2872 (1997)]. Other types of regulatable promoters which may be useful in this context are those which are regulated by a specific physiological state, e.g., temperature, acute phase, a particular differentiation state of the cell, or in replicating cells only.

In another embodiment, the native promoter for the transgene will be used. The native promoter may be preferred when it is desired that expression of the transgene should mimic the native expression. The native promoter may be used when expression of the transgene must be regulated temporally or developmentally, or in a tissue-specific manner, or in response to specific transcriptional stimuli. In a further embodiment, other native expression control elements, such as enhancer elements, polyadenylation sites or Kozak consensus sequences may also be used to mimic the native expression.

Another embodiment of the nucleic acid coding sequence includes a gene operably linked to a tissue-specific promoter. For instance, if expression in skeletal muscle is desired, a promoter active in muscle should be used. These include the promoters from genes encoding skeletal β-actin, myosin light chain 2A, dystrophin, muscle creatine kinase, as well as synthetic muscle promoters with activities higher than naturally-occurring promoters (see Li et al., Nat. Biotech., 17:241-245 (1999)). Examples of promoters that are tissue-specific are known for liver (albumin, Miyatake et al., J. Virol., 71:5124-32 (1997); hepatitis B virus core promoter, Sandig et al., Gene Ther., 3:1002-9 (1996); alpha-fetoprotein (AFP), Arbuthnot et al., Hum. Gene Ther., 7:1503-14 (1996)), bone osteocalcin (Stein et al., Mol. Biol. Rep., 24:185-96 (1997)); bone sialoprotein (Chen et al., J. Bone Miner. Res., 11:654-64 (1996)), lymphocytes (CD2, Hansal et al., J. Immunol., 161:1063-8 (1998); immunoglobulin heavy chain; T cell receptor chain), neuronal such as neuron-specific enolase (NSE) promoter (Andersen et al., Cell. Mol. Neurobiol., 13:503-15 (1993)), neurofilament light-chain gene (Piccioli et al., Proc. Natl. Acad. Sci. USA, 88:5611-5 (1991)), and the neuron-specific vgf gene (Piccioli et al., Neuron, 15:373-84 (1995)), among others.

Optionally, plasmids carrying therapeutically useful transgenes may also include selectable markers or reporter genes may include sequences encoding geneticin, hygromicin or purimycin resistance, among others. Such selectable reporters or marker genes (preferably located outside the viral genome to be rescued by the method of the invention) can be used to signal the presence of the plasmids in bacterial cells, such as ampicillin resistance. Other components of the plasmid may include an origin of replication. Selection of these and other promoters and vector elements are conventional and many such sequences are available [see, e.g., Sambrook et al, and references cited therein].

Provided with the teachings of this invention, the design of such an expression cassette can be made by resort to conventional techniques.

3. Delivery of the Expression Cassette to a Packaging Host Cell

The expression cassette can be carried on any suitable vector, e.g., a plasmid, which is delivered to a host cell. The plasmids useful in this invention may be engineered such that they are suitable for replication and, optionally, integration in prokaryotic cells, mammalian cells, or both. These plasmids (or other vectors carrying the 5′ AAV ITR-heterologous molecule-3′ AAV ITR) contain sequences permitting replication of the expression cassette in eukaryotes and/or prokaryotes and selection markers for these systems. Selectable markers or reporter genes may include sequences encoding geneticin, hygromicin or purimycin resistance, among others. The plasmids may also contain certain selectable reporters or marker genes that can be used to signal the presence of the vector in bacterial cells, such as ampicillin resistance. Other components of the plasmid may include an origin of replication and an amplicon, such as the amplicon system employing the Epstein Barr virus nuclear antigen. This amplicon system, or other similar amplicon components permit high copy episomal replication in the cells. Preferably, the molecule carrying the expression cassette is transfected into the cell, where it may exist transiently. Alternatively, the expression cassette (carrying the 5′ AAV ITR-heterologous molecule-3′ ITR) may be stably integrated into the genome of the host cell, either chromosomally or as an episome. In certain embodiments, the expression cassette be present in multiple copies, optionally in head-to-head, head-to-tail, or tail-to-tail concatamers. Suitable transfection techniques are known and may readily be utilized to deliver the minigene to the host cell.

Generally, when delivering the vector comprising the expression by transfection, the vector is delivered in an amount from about 5 μg to about 100 μg DNA, about 10 μg to about 50 μg DNA to about 1×10⁴ cells to about 1×10¹³ cells, or about 1×10⁵ cells. However, the relative amounts of vector DNA to host cells may be adjusted, taking into consideration such factors as the selected vector, the delivery method and the host cells selected.

B. Rep and Cap Sequences

In addition to the minigene, the host cell contains the sequences which drive expression of a novel AAV capsid protein of the invention (or a capsid protein comprising a fragment thereof) in the host cell and rep sequences of the same source as the source of the AAV ITRs found in the minigene, or a cross-complementing source. The AAV cap and rep sequences may be independently obtained from an AAV source as described above and may be introduced into the host cell in any manner known to one in the art as described above. Additionally, when pseudotyping an AAV vector in a modified AAV, the sequences encoding each of the essential rep proteins may be supplied by different AAV sources (e.g., AAV1, AAV2, AAV3, AAV4, AAV5, AAV6, AAV7, AAV8, AAV9). For example, the rep78/68 sequences may be from AAV2, whereas the rep52/40 sequences may be from AAV8.

In one embodiment, the host cell stably contains the capsid protein under the control of a suitable promoter, such as those described above. Most desirably, in this embodiment, the capsid protein is expressed under the control of a regulatable promoter. In another embodiment, the capsid protein is supplied to the host cell in trans. When delivered to the host cell in trans, the capsid protein may be delivered via a plasmid which contains the sequences necessary to direct expression of the selected capsid protein in the host cell. Most desirably, when delivered to the host cell in trans, the plasmid carrying the capsid protein also carries other sequences required for packaging the rAAV, e.g., the rep sequences.

In another embodiment, the host cell stably contains the rep sequences under the control of a suitable promoter, such as those described above. Most desirably, in this embodiment, the essential rep proteins are expressed under the control of a regulatable promoter. In another embodiment, the rep proteins are supplied to the host cell in trans. When delivered to the host cell in trans, the rep proteins may be delivered via a plasmid which contains the sequences necessary to direct expression of the selected rep proteins in the host cell. Most desirably, when delivered to the host cell in trans, the plasmid carrying the capsid protein also carries other sequences required for packaging the rAAV, e.g., the rep and cap sequences.

Thus, in one embodiment, the rep and cap sequences may be transfected into the host cell on a single nucleic acid molecule and exist stably in the cell as an episome. In another embodiment, the rep and cap sequences are stably integrated into the chromosome of the cell. Another embodiment has the rep and cap sequences transiently expressed in the host cell. For example, a useful nucleic acid molecule for such transfection comprises, from 5′ to 3′, a promoter, an optional spacer interposed between the promoter and the start site of the rep gene sequence, an AAV rep gene sequence, and an AAV cap gene sequence.

Optionally, the rep and/or cap sequences may be supplied on a vector that contains other DNA sequences that are to be introduced into the host cells. For instance, the vector may contain the rAAV construct comprising the minigene. The vector may comprise one or more of the genes encoding the helper functions, e.g., the adenoviral proteins E1, E2a, and E4 ORF6, and the gene for VAI RNA.

Preferably, the promoter used in this construct may be any of the constitutive, regulatable or native promoters known to one of skill in the art or as discussed above. In one embodiment, an AAV P5 promoter sequence is employed. The selection of the AAV to provide any of these sequences does not limit the invention.

In another preferred embodiment, the promoter for rep is a regulatable promoter, such as are discussed above in connection with the transgene regulatory elements. One preferred promoter for rep expression is the T7 promoter. The vector comprising the rep gene regulated by the T7 promoter and the cap gene, is transfected or transformed into a cell which either constitutively or inducibly expresses the T7 polymerase. See International Patent Publication No. WO 98/10088, published Mar. 12, 1998.

The spacer is an optional element in the design of the vector. The spacer is a DNA sequence interposed between the promoter and the rep gene ATG start site. The spacer may have any desired design; that is, it may be a random sequence of nucleotides, or alternatively, it may encode a gene product, such as a marker gene. The spacer may contain genes which typically incorporate start/stop and polyA sites. The spacer may be a non-coding DNA sequence from a prokaryote or eukaryote, a repetitive non-coding sequence, a coding sequence without transcriptional controls or a coding sequence with transcriptional controls. Two exemplary sources of spacer sequences are the phage ladder sequences or yeast ladder sequences, which are available commercially, e.g., from Gibco or Invitrogen, among others. The spacer may be of any size sufficient to reduce expression of the rep78 and rep68 gene products, leaving the rep52, rep40 and cap gene products expressed at normal levels. The length of the spacer may therefore range from about 10 bp to about 10.0 kbp, preferably in the range of about 100 bp to about 8.0 kbp. To reduce the possibility of recombination, the spacer is preferably less than 2 kbp in length; however, the invention is not so limited.

Although the molecule(s) providing rep and cap may exist in the host cell transiently (i.e., through transfection), it is preferred that one or both of the rep and cap proteins and the promoter(s) controlling their expression be stably expressed in the host cell, e.g., as an episome or by integration into the chromosome of the host cell. The methods employed for constructing embodiments of this invention are conventional genetic engineering or recombinant engineering techniques such as those described in the references above. While this specification provides illustrative examples of specific constructs, using the information provided herein, one of skill in the art may select and design other suitable constructs, using a choice of spacers, P5 promoters (which may be from the same source AAV or different from the surrounding sequences, or relocated downstream of the rep expression cassette to control expression), introns, and other elements, including at least one translational start and stop signal, and the optional addition of polyadenylation sites.

In another embodiment of this invention, the rep or cap protein may be provided stably by a host cell.

C. The Helper Functions

The packaging host cell also requires helper functions in order to package the rAAV of the invention. Optionally, these functions may be supplied by a herpesvirus. Most desirably, the necessary helper functions are each provided from a human or non-human primate adenovirus source, such as those described above and/or are available from a variety of sources, including the American Type Culture Collection (ATCC), Manassas, Va. (US). In one currently preferred embodiment, the host cell is provided with and/or contains an E1a gene product, an E1b gene product, an E2a gene product, and/or an E4 ORF6 gene product. The host cell may contain other adenoviral genes such as VAI RNA, but these genes are not required. In a preferred embodiment, no other adenovirus genes or gene functions are present in the host cell.

The adenovirus E1a, E1b, E2a, and/or E4ORF6 gene products, as well as any other desired helper functions, can be provided using any means that allows their expression in a cell. Each of the sequences encoding these products may be on a separate vector, or one or more genes may be on the same vector. The vector may be any vector known in the art or disclosed above, including plasmids, cosmids and viruses. Introduction into the host cell of the vector may be achieved by any means known in the art or as disclosed above, including transfection, infection, electroporation, liposome delivery, membrane fusion techniques, high velocity DNA-coated pellets, viral infection and protoplast fusion, among others. One or more of the adenoviral genes may be stably integrated into the genome of the host cell, stably expressed as episomes, or expressed transiently. The gene products may all be expressed transiently, on an episome or stably integrated, or some of the gene products may be expressed stably while others are expressed transiently. Furthermore, the promoters for each of the adenoviral genes may be selected independently from a constitutive promoter, a regulatable promoter or a native promoter. The promoters may be regulated by a specific physiological state of the organism or cell (i.e., by the differentiation state or in replicating or quiescent cells) or by exogenously added factors, for example.

D. Host Cells and Packaging Cell Lines

The host cell itself may be selected from any biological organism, including prokaryotic (e.g., bacterial) cells, and eukaryotic cells, including, insect cells, yeast cells and mammalian cells. Particularly desirable host cells are selected from among any mammalian species, including, without limitation, cells such as A549, WEHI, 3T3, 10T1/2, BHK, MDCK, COS 1, COS 7, BSC 1, BSC 40, BMT 10, VERO, WI38, HeLa, 293 cells (which express functional adenoviral E1), Saos, C2C12, L cells, HT1080, HepG2 and primary fibroblast, hepatocyte and myoblast cells derived from mammals including human, monkey, mouse, rat, rabbit, and hamster. The selection of the mammalian species providing the cells is not a limitation of this invention; nor is the type of mammalian cell, i.e., fibroblast, hepatocyte, tumor cell, etc. The requirements for the cell used is that it not carry any adenovirus gene other than E1, E2a and/or E4 ORF6; it not contain any other virus gene which could result in homologous recombination of a contaminating virus during the production of rAAV; and it is capable of infection or transfection of DNA and expression of the transfected DNA. In a preferred embodiment, the host cell is one that has rep and cap stably transfected in the cell.

One host cell useful in the present invention is a host cell stably transformed with the sequences encoding rep and cap, and which is transfected with the adenovirus E1, E2a, and E4ORF6 DNA and a construct carrying the minigene as described above. Stable rep and/or cap expressing cell lines, such as B-50 (International Patent Application Publication No. WO 99/15685), or those described in U.S. Pat. No. 5,658,785, may also be similarly employed. Another desirable host cell contains the minimum adenoviral DNA which is sufficient to express E4 ORF6.

The preparation of a host cell according to this invention involves techniques such as assembly of selected DNA sequences. This assembly may be accomplished utilizing conventional techniques. Such techniques include cDNA and genomic cloning, which are well known and are described in Sambrook et al., cited above, use of overlapping oligonucleotide sequences of the adenovirus and AAV genomes, combined with polymerase chain reaction, synthetic methods, and any other suitable methods which provide the desired nucleotide sequence.

Introduction of the molecules (as plasmids or viruses) into the host cell may also be accomplished using techniques known to the skilled artisan and as discussed throughout the specification. In preferred embodiment, standard transfection techniques are used, e.g., CaPO₄ transfection or electroporation, and/or infection by hybrid adenovirus/AAV vectors into cell lines such as the human embryonic kidney cell line HEK 293 (a human kidney cell line containing functional adenovirus E1 genes which provides trans-acting E1 proteins).

Suitable methods for production of an AAV viral particle have been described.

In addition, one desirable method for production of AAV is described in co-owned, US provisional patent application, “Scalable Production Method for AAV”, which is being filed co-currently herewith, and which is hereby incorporated by reference. A method for producing AAV, without requiring cell lysis, is described. The method involves harvesting AAV from the supernatant. In one aspect, the invention involves modifying AAV which do not secrete. For example, AAV having a heparin binding domain which is characterized by having its transduction (infectious) ability blocked by heparin have been found not to secrete in detectable amounts. Examples of such AAV are AAV2 and AAV3. Thus, in one embodiment, the method involves modifying the AAV capsids, the cells, and/or the culture conditions to substantially reduce or eliminate binding between the AAV heparin binding site and the producer cells, thereby allowing the AAV to pass into the supernatant, i.e., media. This method provides supernatant containing high yields of AAV which have a higher degree of purity from cell membranes and intracellular materials, as compared to AAV produced using methods using a cell lysis step.

### Recombinant Viruses and Uses Therefor

In one aspect, a modified AAV of the invention is used for delivery of a therapeutic, immunogenic or vaccinal molecule to a host cell. In one embodiment, the modified AAV of the invention is useful for reducing the immune response and/or toxicity of the modified AAV is substantially lower than the immune response and/or toxicity of the AAV prior to modifying the AAV to ablate heparin binding. The modified AAV of the invention is useful for reducing the immune response and/or toxicity of the modified AAV is substantially lower than the immune response and/or toxicity of the AAV prior to modifying the AAV to alter the RxxR motif.

A. Delivery of Viruses

In another aspect, the present invention provides a method for delivery of a selected heterologous nucleic acid molecule or sequence to a host which involves transfecting or infecting a selected host cell with an AAV viral vector generated with the modified AAV capsids of the invention. Methods for delivery are well known to those of skill in the art and are not a limitation of the present invention.

In one desirable embodiment, the invention provides a method for AAV-mediated delivery of a molecule to a host. This method involves transfecting or infecting a selected host cell with a recombinant viral vector containing a selected molecule under the control of sequences that direct expression thereof and modified AAV capsid proteins.

Optionally, a sample from the host may be first assayed for the presence of antibodies to a selected AAV source (e.g., a serotype). A variety of assay formats for detecting neutralizing antibodies are well known to those of skill in the art. The selection of such an assay is not a limitation of the present invention. See, e.g., Fisher et al, Nature Med., 3(3):306-312 (March 1997) and W. C. Manning et al, Human Gene Therapy, 9:477-485 (Mar. 1, 1998). The results of this assay may be used to determine which AAV vector containing capsid proteins of a particular source are preferred for delivery, e.g., by the absence of neutralizing antibodies specific for that capsid source.

In one aspect of this method, the delivery of vector with modified AAV capsid proteins of the invention may precede or follow delivery of a gene via a vector with a different AAV capsid protein. Thus, gene delivery via AAV vectors may be used for repeat gene delivery to a selected host cell. Desirably, subsequently administered AAV vectors carry the same transgene as the first AAV vector, but the subsequently administered vectors contain capsid proteins of sources (and preferably, different serotypes) which differ from the first vector.

Optionally, multiple AAV vectors can be used to deliver large genes or multiple genes by co-administration of AAV vectors concatamerize in vivo to form a single vector genome. In such an embodiment, a first AAV may carry an expression cassette which expresses a single gene (or a subunit thereof) and a second AAV may carry an expression cassette which expresses a second gene (or a different subunit) for co-expression in the host cell. A first AAV may carry an expression cassette which is a first piece of a polycistronic construct (e.g., a promoter and transgene, or subunit) and a second AAV may carry an expression cassette which is a second piece of a polycistronic construct (e.g., gene or subunit and a polyA sequence). These two pieces of a polycistronic construct concatamerize in vivo to form a single vector genome that co-expresses the genes delivered by the first and second AAV. In such embodiments, the modified AAV vector carrying the first expression cassette and the modified AAV vector carrying the second expression cassette can be delivered in a single pharmaceutical composition. In other embodiments, the two or more modified AAV vectors are delivered as separate pharmaceutical compositions which can be administered substantially simultaneously, or shortly before or after one another.

The above-described recombinant vectors may be delivered to host cells according to published methods. The modified AAV, preferably suspended in a physiologically compatible carrier, may be administered to a human or non-human mammalian patient. Suitable carriers may be readily selected by one of skill in the art in view of the indication for which the transfer virus is directed. For example, one suitable carrier includes saline, which may be formulated with a variety of buffering solutions (e.g., phosphate buffered saline). Other exemplary carriers include sterile saline, lactose, sucrose, calcium phosphate, gelatin, dextran, agar, pectin, peanut oil, sesame oil, and water. The selection of the carrier is not a limitation of the present invention.

Optionally, the compositions of the invention may contain, in addition to the modified AAV and carrier(s), other conventional pharmaceutical ingredients, such as preservatives, or chemical stabilizers. Suitable exemplary preservatives include chlorobutanol, potassium sorbate, sorbic acid, sulfur dioxide, propyl gallate, the parabens, ethyl vanillin, glycerin, phenol, and parachlorophenol. Suitable chemical stabilizers include gelatin and albumin.

The vectors are administered in sufficient amounts to transfect the cells and to provide sufficient levels of gene transfer and expression to provide a therapeutic benefit without undue adverse effects, or with medically acceptable physiological effects, which can be determined by those skilled in the medical arts. Conventional and pharmaceutically acceptable routes of administration include, but are not limited to, direct delivery to a desired organ (e.g., the liver (optionally via the hepatic artery) or lung), oral, inhalation, intranasal, intratracheal, intraarterial, intraocular, intracochlear, intravenous, intramuscular, subcutaneous, intradermal, and other parental routes of administration. Routes of administration may be combined, if desired.

Dosages of the viral vector will depend primarily on factors such as the condition being treated, the age, weight and health of the patient, and may thus vary among patients. For example, a therapeutically effective human dosage of the viral vector is generally in the range of from about 0.1 mL to about 100 mL of solution containing concentrations of from about 1×10⁹ to 1×10¹⁶ genomes virus vector. A preferred human dosage for delivery to large organs (e.g., liver, muscle, heart and lung) may be about 5×10¹⁰ to 5×10¹³ AAV genomes per 1 kg, at a volume of about 1 to 100 mL. A preferred dosage for delivery to eye or ear (cochlea) is about 5×10⁹ to 5×10¹² genome copies, at a volume of about 0.1 mL to 1 mL. The dosage will be adjusted to balance the therapeutic benefit against any side effects and such dosages may vary depending upon the therapeutic application for which the recombinant vector is employed. The levels of expression of the transgene can be monitored to determine the frequency of dosage resulting in viral vectors, preferably AAV vectors containing the minigene. Optionally, dosage regimens similar to those described for therapeutic purposes may be utilized for immunization using the compositions of the invention.

Examples of therapeutic products and immunogenic products for delivery by the modified AAV-containing vectors of the invention are provided below. These vectors may be used for a variety of therapeutic or vaccinal regimens, as described herein. Additionally, these vectors may be delivered in combination with one or more other vectors or active ingredients in a desired therapeutic and/or vaccinal regimen.

B. Therapeutic Products

Useful therapeutic products encoded by the nucleic acid molecule carried on the expression cassette include hormones and growth and differentiation factors including, without limitation, insulin, glucagon, growth hormone (GH), parathyroid hormone (PTH), growth hormone releasing factor (GRF), follicle stimulating hormone (FSH), luteinizing hormone (LH), human chorionic gonadotropin (hCG), vascular endothelial growth factor (VEGF), angiopoietins, angiostatin, granulocyte colony stimulating factor (GCSF), erythropoietin (EPO), connective tissue growth factor (CTGF), basic fibroblast growth factor (bFGF), acidic fibroblast growth factor (aFGF), epidermal growth factor (EGF), platelet-derived growth factor (PDGF), insulin growth factors I and II (IGF-I and IGF-II), any one of the transforming growth factor α superfamily, including TGFα, activins, inhibins, or any of the bone morphogenic proteins (BMP) BMPs 1-15, any one of the heregluin/neuregulin/ARIA/neu differentiation factor (NDF) family of growth factors, nerve growth factor (NGF), brain-derived neurotrophic factor (BDNF), neurotrophins NT-3 and NT-4/5, ciliary neurotrophic factor (CNTF), glial cell line derived neurotrophic factor (GDNF), neurturin, agrin, any one of the family of semaphorins/collapsins, netrin-1 and netrin-2, hepatocyte growth factor (HGF), ephrins, noggin, sonic hedgehog and tyrosine hydroxylase.

Other useful transgene products include proteins that regulate the immune system including, without limitation, cytokines and lymphokines such as thrombopoietin (TPO), interleukins (IL) IL-1 through IL-25 (including, e.g., IL-2, IL-4, IL-12 and IL-18), monocyte chemoattractant protein, leukemia inhibitory factor, granulocyte-macrophage colony stimulating factor, Fas ligand, tumor necrosis factors α and β, interferons α, β, and γ, stem cell factor, flk-2/flt3 ligand. Gene products produced by the immune system are also useful in the invention. These include, without limitations, immunoglobulins IgG, IgM, IgA, IgD and IgE, chimeric immunoglobulins, humanized antibodies, single chain antibodies, T cell receptors, chimeric T cell receptors, single chain T cell receptors, class I and class II MHC molecules, as well as engineered immunoglobulins and MHC molecules. Useful gene products also include complement regulatory proteins such as complement regulatory proteins, membrane cofactor protein (MCP), decay accelerating factor (DAF), CR1, CF2 and CD59.

Still other useful gene products include any one of the receptors for the hormones, growth factors, cytokines, lymphokines, regulatory proteins and immune system proteins. The invention encompasses receptors for cholesterol regulation and/or lipid modulation, including the low density lipoprotein (LDL) receptor, high density lipoprotein (HDL) receptor, the very low density lipoprotein (VLDL) receptor, and scavenger receptors. The invention also encompasses gene products such as members of the steroid hormone receptor superfamily including glucocorticoid receptors and estrogen receptors, Vitamin D receptors and other nuclear receptors. In addition, useful gene products include transcription factors such as jun, fos, max, mad, serum response factor (SRF), AP-1, AP2, myb, MyoD and myogenin, ETS-box containing proteins, TFE3, E2F, ATF1, ATF2, ATF3, ATF4, ZF5, NFAT, CREB, HNF-4, C/EBP, SP1, CCAAT-box binding proteins, interferon regulation factor (IRF-1), Wilms tumor protein, ETS-binding protein, STAT, GATA-box binding proteins, e.g., GATA-3, and the forkhead family of winged helix proteins.

Other useful gene products include, carbamoyl synthetase 1, ornithine transcarbamylase, arginosuccinate synthetase, arginosuccinate lyase, arginase, fumarylacetacetate hydrolase, phenylalanine hydroxylase, alpha-1 antitrypsin, glucose-6-phosphatase, porphobilinogen deaminase, cystathione beta-synthase, branched chain ketoacid decarboxylase, albumin, isovaleryl-coA dehydrogenase, propionyl CoA carboxylase, methyl malonyl CoA mutase, glutaryl CoA dehydrogenase, insulin, beta-glucosidase, pyruvate carboxylate, hepatic phosphorylase, phosphorylase kinase, glycine decarboxylase, H-protein, T-protein, a cystic fibrosis transmembrane regulator (CFTR) sequence, and a dystrophin gene product [e.g., a mini- or micro-dystrophin]. Still other useful gene products include enzymes such as may be useful in enzyme replacement therapy, which is useful in a variety of conditions resulting from deficient activity of enzyme. For example, enzymes that contain mannose-6-phosphate may be utilized in therapies for lysosomal storage diseases (e.g., a suitable gene includes that encoding β-glucuronidase (GUSB)).

Still other useful gene products include those used for treatment of hemophilia, including hemophilia B (including Factor IX) and hemophilia A (including Factor VIII and its variants, such as the light chain and heavy chain of the heterodimer and the B-deleted domain; U.S. Pat. No. 6,200,560 and U.S. Pat. No. 6,221,349). The Factor VIII gene codes for 2351 amino acids and the protein has six domains, designated from the amino to the terminal carboxy terminus as A1-A2-B-A3-C1-C2 [Wood et al, Nature, 312:330 (1984); Vehar et al., Nature 312:337 (1984); and Toole et al, Nature, 342:337 (1984)]. Human Factor VIII is processed within the cell to yield a heterodimer primarily comprising a heavy chain containing the A1, A2 and B domains and a light chain containing the A3, C1 and C2 domains. Both the single chain polypeptide and the heterodimer circulate in the plasma as inactive precursors, until activated by thrombin cleavage between the A2 and B domains, which releases the B domain and results in a heavy chain consisting of the A1 and A2 domains. The B domain is deleted in the activated procoagulant form of the protein. Additionally, in the native protein, two polypeptide chains (“a” and “b”), flanking the B domain, are bound to a divalent calcium cation.

In some embodiments, the minigene comprises first 57 base pairs of the Factor VIII heavy chain which encodes the 10 amino acid signal sequence, as well as the human growth hormone (hGH) polyadenylation sequence. In alternative embodiments, the minigene further comprises the A1 and A2 domains, as well as 5 amino acids from the N-terminus of the B domain, and/or 85 amino acids of the C-terminus of the B domain, as well as the A3, C1 and C2 domains. In yet other embodiments, the nucleic acids encoding Factor VIII heavy chain and light chain are provided in a single minigene separated by 42 nucleic acids coding for 14 amino acids of the B domain [U.S. Pat. No. 6,200,560].

As used herein, a therapeutically effective amount is an amount of AAV vector that produces sufficient amounts of Factor VIII to decrease the time it takes for a subject's blood to clot. Generally, severe hemophiliacs having less than 1% of normal levels of Factor VIII have a whole blood clotting time of greater than 60 minutes as compared to approximately 10 minutes for non-hemophiliacs.

The present invention is not limited to any specific Factor VIII sequence. Many natural and recombinant forms of Factor VIII have been isolated and generated. Examples of naturally occurring and recombinant forms of Factor VII can be found in the patent and scientific literature including, U.S. Pat. No. 5,563,045, U.S. Pat. No. 5,451,521, U.S. Pat. No. 5,422,260, U.S. Pat. No. 5,004,803, U.S. Pat. No. 4,757,006, U.S. Pat. No. 5,661,008, U.S. Pat. No. 5,789,203, U.S. Pat. No. 5,681,746, U.S. Pat. No. 5,595,886, U.S. Pat. No. 5,045,455, U.S. Pat. No. 5,668,108, U.S. Pat. No. 5,633,150, U.S. Pat. No. 5,693,499, U.S. Pat. No. 5,587,310, U.S. Pat. No. 5,171,844, U.S. Pat. No. 5,149,637, U.S. Pat. No. 5,112,950, U.S. Pat. No. 4,886,876; International Patent Publication Nos. WO 94/11503, WO 87/07144, WO 92/16557, WO 91/09122, WO 97/03195, WO 96/21035, and WO 91/07490; European Patent Application Nos. EP 0 672 138, EP 0 270 618, EP 0 182 448, EP 0 162 067, EP 0 786 474, EP 0 533 862, EP 0 506 757, EP 0 874 057, EP 0 795 021, EP 0 670 332, EP 0 500 734, EP 0 232 112, and EP 0 160 457; Sanberg et al., XXth Int. Congress of the World Fed. Of Hemophilia (1992), and Lind et al., Eur. J. Biochem., 232:19 (1995).

Nucleic acids sequences coding for the above-described Factor VIII can be obtained using recombinant methods or by deriving the sequence from a vector known to include the same. Furthermore, the desired sequence can be isolated directly from cells and tissues containing the same, using standard techniques, such as phenol extraction and PCR of cDNA or genomic DNA [See, e.g., Sambrook et al]. Nucleotide sequences can also be produced synthetically, rather than cloned. The complete sequence can be assembled from overlapping oligonucleotides prepared by standard methods and assembled into a complete coding sequence [See, e.g., Edge, Nature 292:757 (1981); Nambari et al, Science, 223:1299 (1984); and Jay et al, J. Biol. Chem. 259:6311 (1984).

Furthermore, the invention is not limited to human Factor VIII. Indeed, it is intended that the present invention encompass Factor VIII from animals other than humans, including but not limited to companion animals (e.g., canine, felines, and equines), livestock (e.g., bovines, caprines and ovines), laboratory animals, marine mammals, large cats, etc.

The AAV vectors may contain a nucleic acid coding for fragments of Factor VIII which is itself not biologically active, yet when administered into the subject improves or restores the blood clotting time. For example, as discussed above, the Factor VIII protein comprises two polypeptide chains: a heavy chain and a light chain separated by a B-domain which is cleaved during processing. As demonstrated by the present invention, co-tranducing recipient cells with the Factor VIII heavy and light chains leads to the expression of biologically active Factor VIII. Because most hemophiliacs contain a mutation or deletion in only one of the chains (e.g., heavy or light chain), it may be possible to administer only the chain defective in the patient to supply the other chain.

Other useful gene products include non-naturally occurring polypeptides, such as chimeric or hybrid polypeptides having a non-naturally occurring amino acid sequence containing insertions, deletions or amino acid substitutions. For example, single-chain engineered immunoglobulins could be useful in certain immunocompromised patients. Other types of non-naturally occurring gene sequences include antisense molecules and catalytic nucleic acids, such as ribozymes, which could be used to reduce overexpression of a target.

Reduction and/or modulation of expression of a gene is particularly desirable for treatment of hyperproliferative conditions characterized by hyperproliferating cells, as are cancers and psoriasis. Target polypeptides include those polypeptides which are produced exclusively or at higher levels in hyperproliferative cells as compared to normal cells. Target antigens include polypeptides encoded by oncogenes such as myb, myc, fyn, and the translocation gene bcr/abl, ras, src, P53, neu, trk and EGRF. In addition to oncogene products as target antigens, target polypeptides for anti-cancer treatments and protective regimens include variable regions of antibodies made by B cell lymphomas and variable regions of T cell receptors of T cell lymphomas which, in some embodiments, are also used as target antigens for autoimmune disease. Other tumor-associated polypeptides can be used as target polypeptides such as polypeptides which are found at higher levels in tumor cells including the polypeptide recognized by monoclonal antibody 17-1A and folate binding polypeptides.

Other suitable therapeutic polypeptides and proteins include those which may be useful for treating individuals suffering from autoimmune diseases and disorders by conferring a broad based protective immune response against targets that are associated with autoimmunity including cell receptors and cells which produce “self”-directed antibodies. T cell mediated autoimmune diseases include Rheumatoid arthritis (RA), multiple sclerosis (MS), Sjögren's syndrome, sarcoidosis, insulin dependent diabetes mellitus (IDDM), autoimmune thyroiditis, reactive arthritis, ankylosing spondylitis, scleroderma, polymyositis, dermatomyositis, psoriasis, vasculitis, Wegener's granulomatosis, Crohn's disease and ulcerative colitis. Each of these diseases is characterized by T cell receptors (TCRs) that bind to endogenous antigens and initiate the inflammatory cascade associated with autoimmune diseases.

C. Immunogenic Transgenes

Suitably, the AAV vectors of the invention avoid the generation of immune responses to the AAV capsid sequences. However, these vectors may nonetheless be formulated in a manner that permits the expression of a transgene carried by the vectors to induce an immune response to a selected antigen. For example, in order to promote an immune response, the gene product may be expressed from a constitutive promoter, the vector can be adjuvanted as described herein, and/or the vector can be put into degenerating tissue.

Examples of suitable antigenic and immunogenic products for delivery by the modified AAV-containing vectors of the invention are provided below. These vectors may be used for a variety of immunogenic or vaccinal regimens, as described herein. Additionally, these vectors may be delivered in combination with one or more other vectors or active ingredients in a desired immunomodulatory and/or vaccinal regimen. See, e.g., the prime-boost regimens utilized AAV vectors described in International application no. PCT/US2005/014556, filed 27 Apr. 2005.

Suitably, the AAV vectors of the invention enhance cellular (i.e., T-cell) immune responses to the AAV contained within the vector. However, these vectors may nonetheless be formulated in a manner that permits the expression of a transgene carried by the vectors to induce an immune response to a selected antigen. For example, in order to promote an immune response, the transgene may be expressed from a constitutive promoter, the vector can be adjuvanted as described herein, and/or the vector can be put into degenerating tissue.

Examples of suitable immunogenic and antigenic products include those derived from a variety of viral families. Examples of desirable viral families against which an immune response would be desirable include, the picornavirus family, which includes the genera rhinoviruses, which are responsible for about 50% of cases of the common cold; the genera enteroviruses, which include polioviruses, coxsackieviruses, echoviruses, and human enteroviruses such as hepatitis A virus; and the genera apthoviruses, which are responsible for foot and mouth diseases, primarily in non-human animals. Within the picornavirus family of viruses, target antigens include the VP1, VP2, VP3, VP4, and VPG. Other viral families include the astroviruses and the calcivirus family. The calcivirus family encompasses the Norwalk group of viruses, which are an important causative agent of epidemic gastroenteritis. Still another viral family desirable for use in targeting antigens for inducing immune responses in humans and non-human animals is the togavirus family, which includes the genera alphavirus, which include Sindbis viruses, RossRiver virus, and Venezuelan, Eastern & Western Equine encephalitis, and rubivirus, including Rubella virus. The flaviviridae family includes dengue, yellow fever, Japanese encephalitis, St. Louis encephalitis and tick borne encephalitis viruses. Other target antigens may be generated from the Hepatitis C or the coronavirus family, which includes a number of non-human viruses such as infectious bronchitis virus (poultry), porcine transmissible gastroenteric virus (pig), porcine hemagglutinatin encephalomyelitis virus (pig), feline infectious peritonitis virus (cat), feline enteric coronavirus (cat), canine coronavirus (dog), and human respiratory coronaviruses, which may cause the common cold and/or non-A, B or C hepatitis, and which include the putative cause of sudden acute respiratory syndrome (SARS). Within the coronavirus family, target antigens include the E1 (also called M or matrix protein), E2 (also called S or Spike protein), E3 (also called HE or hemagglutin-elterose) glycoprotein (not present in all coronaviruses), or N (nucleocapsid). Still other antigens may be targeted against the arterivirus family and the rhabdovirus family. The rhabdovirus family includes the genera vesiculovirus (e.g., Vesicular Stomatitis Virus), and the general lyssavirus (e.g., rabies). Within the rhabdovirus family, suitable antigens may be derived from the G protein or the N protein. The family filoviridae, which includes hemorrhagic fever viruses such as Marburg and Ebola virus may be a suitable source of antigens. The paramyxovirus family includes parainfluenza Virus Type 1, parainfluenza Virus Type 3, bovine parainfluenza Virus Type 3, rubulavirus (mumps virus, parainfluenza Virus Type 2, parainfluenza virus Type 4, Newcastle disease virus (chickens), rinderpest, morbillivirus, which includes measles and canine distemper, and pneumovirus, which includes respiratory syncytial virus. The influenza virus is classified within the family orthomyxovirus and is a suitable source of antigen (e.g., the HA protein, the N1 protein). The bunyavirus family includes the genera bunyavirus (California encephalitis, La Crosse), phlebovirus (Rift Valley Fever), hantavirus (puremala is a hemahagin fever virus), nairovirus (Nairobi sheep disease) and various unassigned bungaviruses. The arenavirus family provides a source of antigens against LCM and Lassa fever virus. Another source of antigens is the bornavirus family. The reovirus family includes the genera reovirus, rotavirus (which causes acute gastroenteritis in children), orbiviruses, and cultivirus (Colorado Tick fever, Lebombo (humans), equine encephalosis, blue tongue). The retrovirus family includes the sub-family oncorivirinal which encompasses such human and veterinary diseases as feline leukemia virus, HTLVI and HTLVII, lentivirinal (which includes HIV, simian immunodeficiency virus, feline immunodeficiency virus, equine infectious anemia virus, and spumavirinal). The papovavirus family includes the sub-family polyomaviruses (BKU and JCU viruses) and the sub-family papillomavirus (associated with cancers or malignant progression of papilloma). The adenovirus family includes viruses (EX, AD7, ARD, O.B.) which cause respiratory disease and/or enteritis. The parvovirus family includes feline parvovirus (feline enteritis), feline panleucopeniavirus, canine parvovirus, and porcine parvovirus. The herpesvirus family includes the sub-family alphaherpesvirinae, which encompasses the genera simplexvirus (HSVI, HSVII), varicellovirus (pseudorabies, varicella zoster) and the sub-family betaherpesvirinae, which includes the genera cytomegalovirus (HCMV, muromegalovirus) and the sub-family gammaherpesvirinae, which includes the genera lymphocryptovirus, EBV (Burkitts lymphoma), human herpesviruses 6A, 6B and 7, Kaposi's sarcoma-associated herpesvirus and cercopithecine herpesvirus (B virus), infectious rhinotracheitis, Marek's disease virus, and rhadinovirus. The poxvirus family includes the sub-family chordopoxvirinae, which encompasses the genera orthopoxvirus (Variola major (Smallpox) and Vaccinia (Cowpox)), parapoxvirus, avipoxvirus, capripoxvirus, leporipoxvirus, suipoxvirus, and the sub-family entomopoxvirinae. The hepadnavirus family includes the Hepatitis B virus. One unclassified virus which may be suitable source of antigens is the Hepatitis delta virus, Hepatitis E virus, and prions. Another virus which is a source of antigens is Nipan Virus. Still other viral sources may include avian infectious bursal disease virus and porcine respiratory and reproductive syndrome virus. The alphavirus family includes equine arteritis virus and various Encephalitis viruses.

Other immunogens include those which are useful to immunize a human or non-human animal against other pathogens including bacteria, fungi, parasitic microorganisms or multicellular parasites which infect human and non-human vertebrates, or from a cancer cell or tumor cell. Examples of bacterial pathogens include pathogenic gram-positive cocci include pneumococci; staphylococci (and the toxins produced thereby, e.g., enterotoxin B); and streptococci. Pathogenic gram-negative cocci include meningococcus; gonococcus. Pathogenic enteric gram-negative bacilli include enterobacteriaceae; pseudomonas, acinetobacteria and eikenella; melioidosis; salmonella; shigella; haemophilus; moraxella; H. ducreyi (which causes chancroid); brucella species (brucellosis); Francisella tularensis (which causes tularemia); Yersinia pestis (plague) and other yersinia (pasteurella); streptobacillus moniliformis and spirillum; Gram-positive bacilli include listeria monocytogenes; erysipelothrix rhusiopathiae; Corynebacterium diphtheria (diphtheria); cholera; B. anthracis (anthrax); donovanosis (granuloma inguinale); and bartonellosis. Diseases caused by pathogenic anaerobic bacteria include tetanus; botulism (Clostridum botulinum and its toxin); Clostridium perfringens and its epsilon toxin; other clostridia; tuberculosis; leprosy; and other mycobacteria. Pathogenic spirochetal diseases include syphilis; treponematoses: yaws, pinta and endemic syphilis; and leptospirosis. Other infections caused by higher pathogen bacteria and pathogenic fungi include glanders (Burkholderia mallei); actinomycosis; nocardiosis; cryptococcosis, blastomycosis, histoplasmosis and coccidioidomycosis; candidiasis, aspergillosis, and mucormycosis; sporotrichosis; paracoccidiodomycosis, petriellidiosis, torulopsosis, mycetoma and chromomycosis; and dermatophytosis. Rickettsial infections include Typhus fever, Rocky Mountain spotted fever, Q fever (Coxiella burnetti), and Rickettsial pox. Examples of mycoplasma and chlamydial infections include: mycoplasma pneumoniae; lymphogranuloma venereum; psittacosis; and perinatal chlamydial infections. Pathogenic eukaryotes encompass pathogenic protozoans and helminths and infections produced thereby include: amebiasis; malaria; leishmaniasis; trypanosomiasis; toxoplasmosis; Pneumocystis carinii; Trichans; Toxoplasma gondii; babesiosis; giardiasis; trichinosis; filariasis; schistosomiasis; nematodes; trematodes or flukes; and cestode (tapeworm) infections.

Many of these organisms and/or the toxins produced thereby have been identified by the Centers for Disease Control [(CDC), Department of Heath and Human Services, USA], as agents which have potential for use in biological attacks. For example, some of these biological agents, include, Bacillus anthracis (anthrax), Clostridium botulinum and its toxin (botulism), Yersinia pestis (plague), variola major (smallpox), Francisella tularensis (tularemia), and viral hemorrhagic fevers [filoviruses (e.g., Ebola, Marburg], and arenaviruses [e.g., Lassa, Machupo]), all of which are currently classified as Category A agents; Coxiella burnetti (Q fever); Brucella species (brucellosis), Burkholderia mallei (glanders), Burkholderia pseudomallei (meloidosis), Ricinus communis and its toxin (ricin toxin); Clostridium perfringens and its toxin (epsilon toxin), Staphylococcus species and their toxins (enterotoxin B), Chlamydia psittaci (psittacosis), water safety threats (e.g., Vibrio cholerae, Crytosporidium parvum), Typhus fever (Richettsia powazekii), and viral encephalitis (alphaviruses, e.g., Venezuelan equine encephalitis; eastern equine encephalitis; western equine encephalitis); all of which are currently classified as Category B agents; and Nipan virus and hantaviruses, which are currently classified as Category C agents. In addition, other organisms, which are so classified or differently classified, may be identified and/or used for such a purpose in the future. It will be readily understood that the viral vectors and other constructs described herein are useful to deliver antigens from these organisms, viruses, their toxins or other by-products, which will prevent and/or treat infection or other adverse reactions with these biological agents.

Administration of the vectors of the invention to deliver immunogens against the variable region of the T cells elicit an immune response including CTLs to eliminate those T cells. In rheumatoid arthritis (RA), several specific variable regions of TCRs which are involved in the disease have been characterized. These TCRs include V-3, V-14, V-17 and V-17. Thus, delivery of a nucleic acid sequence that encodes at least one of these polypeptides will elicit an immune response that will target T cells involved in RA. In multiple sclerosis (MS), several specific variable regions of TCRs which are involved in the disease have been characterized. These TCRs include V-7 and V-10. Thus, delivery of a nucleic acid sequence that encodes at least one of these polypeptides will elicit an immune response that will target T cells involved in MS. In scleroderma, several specific variable regions of TCRs which are involved in the disease have been characterized. These TCRs include V-6, V-8, V-14 and V-16, V-3C, V-7, V-14, V-15, V-16, V-28 and V-12. Thus, delivery of a nucleic acid molecule that encodes at least one of these polypeptides will elicit an immune response that will target T cells involved in scleroderma.

Thus, a modified rAAV viral vector of the invention provides an efficient gene transfer vehicle which can deliver a selected transgene to a selected host cell in vivo or ex vivo even where the organism has neutralizing antibodies to one or more AAV sources. In one embodiment, the rAAV and the cells are mixed ex vivo; the infected cells are cultured using conventional methodologies; and the transduced cells are re-infused into the patient.

Thus, a modified AAV of the invention provides an efficient gene transfer vehicle which can deliver a selected transgene to a selected host cell in vivo or ex vivo even where the organism has neutralizing antibodies to one or more AAV sources. In one embodiment, the AAV and the cells are mixed ex vivo, the infected cells are cultured using conventional methodologies; and the transduced cells are re-infused into the patient.

These compositions are particularly well suited to gene delivery for therapeutic purposes and for immunization, including inducing protective immunity. Further, the compositions of the invention may also be used for production of a desired gene product in vitro. For in vitro production, a desired product (e.g., a protein) may be obtained from a desired culture following transfection of host cells with a AAV containing the molecule encoding the desired product and culturing the cell culture under conditions which permit expression. The expressed product may then be purified and isolated, as desired. Suitable techniques for transfection, cell culturing, purification, and isolation are known to those of skill in the art.

### EXAMPLES

The studies provided herein indicate that the critical path to activation of T cells to capsid is not a function of MHC class I restriction but rather dependent on the binding of capsid to heparan sulfate glycoprotein (or heparin). In the present specification, it is shown in both mice and nonhuman primates that engineered or natural variants of AAV that do not bind heparin are less likely to activate T cells to the capsid. All currently known members of AAVs from Clades A, C, D, E and F are missing the RxxR [SEQ ID NO: 2] motif. Further, the members of those clades that have been studied to date do not bind heparin with the avidity of AAV2 [Halbert, C. L., et al., J Virol 75, 6615-24 (2001)], including AAV8 and AAV9 which demonstrate superior transduction profiles to liver and heart, respectively. In fact, some members of the Clade B family such as hu.13, which are virtually identical to AAV2 except in the heparin binding domain, retain levels of in vivo gene transfer similar to AAV2 without the problem of capsid T cells.

The mechanism by which heparin binding directs the activation of T cells to the capsid is unclear. HSP has been shown by others to bind to dendritic cells and promote their activation. It is postulated by the inventors that binding of capsid to HSP shuttles the virion into a dendritic cell pathway that leads to its processing and MHC class I presentation. Pathways by which this occurs begin with endocytotic or phagocytotic uptake followed by a series of proteolytic steps and eventual loading of peptides onto MHC class I complexes. Where along these pathways HSP binding promotes the process of cross-presentation is unclear. It is interesting that these pathways are independent of vector transduction since heparin binding deficient virions of various Clades retain excellent transduction profiles. Furthermore heparin binding is not necessary for T and B cell responses to the transgene; the highest T cell responses we observe to transgene products are from the non heparin binders AAV7 and 8. AAV presents an interesting divergence of MHC class I pathways directed by the structure of its capsid.

The following examples show the mapping of a T cell epitope to the RxxR [SEQ ID NO: 2] domain in the AAV2 capsid [SEQ ID NO: 3]. Exemplary methods of constructing modified AAV having an ablated RxxR domain, or an artificially inserted RxxR domain are illustrated. Also illustrated are methods of delivering such constructs to animals, including mammals.

Table 1 provides a description of AAV isolates and mutants referenced throughout this specification. The capsid sequences of the isolates, AAV2 [SEQ ID NO: 3], hu. 51 [SEQ ID NO: 7], hu.13 [SEQ ID NO: 12], AAV8 [SEQ ID NO: 13] and AAV7 [SEQ ID NO: 14] and the AAV8RQNR mutant of AAV8 [SEQ ID NO: 13], the hu.29R mutant of hu.29 [SEQ ID NO: 15], and the AAV2HSPG-mutant of AAV2 [SEQ ID NO: 3] are previously published but also provided in the Sequence Listing for convenience. The name of the isolate or mutant, its phylogenetic clade, amino acid sequence at AAV2-parallel RxxR [SEQ ID NO: 2] motif, heparin column binding affinity (+, specific binding; −, no binding) and the distance from AAV2 outside of the RxxR domain is provided. The distance is given in number of residues difference outside of RxxR when compared to AAV2. For Clade B members, amino acid differences are presented with their coordinates.

TABLE 1

| | | | | |
|---|---|---|---|---|
| | SEQ ID | | | |
| | NO: | | | |
| | (based | | | |
| AAV | on | | | Distance from |
| isolate/ | native | | | AAV2 outside |
| mutant | seq) | AAV Clade | RxxR domain | RxxR |

| | | | | |
|---|---|---|---|---|
| AAV2 | 3 | B | RGNR/SEQ ID NO 16 | 0/738 |
| hu.51 | 7 | B | RGNR/SEQ ID NO 16 | 4/738 (G133, |
| | | | | G423, T447, N529) |
| AAV8RQNR | 13 | E | RQNR/SEQ ID NO 16 | 119/738 |
| hu.29R | 15 | B | SGNT/SEQ ID NO 18 | 5/738 (A151, S162, |
| | | | | N164, S179, P547 |
| hu13 | 12 | B | GGNT/SEQ ID NO 19 | 2/738 (A151, S205) |
| AAV2HSPG- | 3 | B | SGNT/SEQ ID NO 18 | 0/738 |
| AAV8 | 13 | E | QQNT/SEQ ID NO 20 | 119/738 |
| AAV7 | 14 | D | AANT/SEQ ID NO 21 | 127/738 |

### Example 1

### Activation of T Cells in Mice Following AAV Administration

In the current study, mice (C57BL/6 and Balb/C) were injected IM with 10¹¹ genome containing particles (GC) of AAV2, 2/7 and 2/8 and were evaluated for activation of T cells to capsid proteins by Enzyme-linked ImmunoSPOT (ELISPOT) (all vectors contain the same genomes based on AAV2 encapsulated with different capsids). Splenocytes were stimulated with pooled peptides spanning the entire VP1 capsid as well as the mapped dominant peptides. High level capsid specific T cells were detected to vectors based on AAV2 and a number of phylogenetically related AAV variants. However, vectors from other AAV clades [Gao, G. et al. J Virol 78, 6381-8 (2004)] such asAAV8 [Gao, G. P. et al. Proc Natl Acad Sci USA 99, 11854-9 (2002)] did not lead to activation of capsid specific T cells.

A. Construction of AAV Vectors

The packaging plasmid used express AAV2 rep cloned in cis with the particular cap gene as described [Gao, G. P. et al. Proc Natl Acad Sci USA 99, 11854-9 (2002)]. All natural isolates were previously described [Gao, G. et al., J Virol 78, 6381-8 (2004); G. Gao et al, (2002); Gao, G. et al. Proc Natl Acad Sci USA 100, 6081-6 (2003)]. High titer vector preparations were produced by triple-transfection and purified by three sedimentation rounds on a CsCl gradient.

B. Mouse Immunization

Male C57Bl/6 and Balb/C were obtained from Charles River Laboratories. Animals were injected with 10¹¹ GC by intramuscular injection in the hind limb at two injection sites. The mouse immunization studies were performed both with 1) a nuclear targeted LacZ transgene driven from an enhanced chicken β-actin promotor with a polyadenylation signaling sequence from the bovine growth hormone and AAV vectors, and 2) a human α-1 antitrypsin (A1AT) gene driven from the enhanced chicken β-actin promoter. Gene transfer efficiency experiments were performed with the A1AT vectors.

INF-γ ELISPOT assays were performed using previously described protocols for mice [Simmons, G. et al. Virology 318, 224-30 (2004); Zhi, Y. et al. Virology 335, 34-45 (2005)]. Peptide libraries derived from the VP1 of AAV2, 7 or 8 proteins were synthesized as 15-mers with 10-amino-acid overlap with the proceeding peptide (Mimotopes) and dissolved in DMSO at approximately 100 mg/ml.

Balb/c mice experiments were done with the following H2^{d} restricted epitopes: VPQYGYLTL, SEQ ID NO: 22 (AAV2) and IPQYGYLTL, SEQ ID NO: 1 (AAV7 and AAV8). Peptides were used at the concentration of 2 μg/ml in all experiments and DMSO concentrations were kept below 0.1% (v/v) in all final assay mixtures. Spots were counted with an ELISPOT reader (AID). Besides peptide stimulation, a no peptide condition and non specific stimulation with SEB and PMA/ionomycin controls were performed. Spot numbers were normalized for cell numbers with the PMA/ionomycin values in order to account for slight variation in cell density in the ELISPOT assay.

C. Detection of AAV2 Capsid-Specific T Cells in Mice Studies

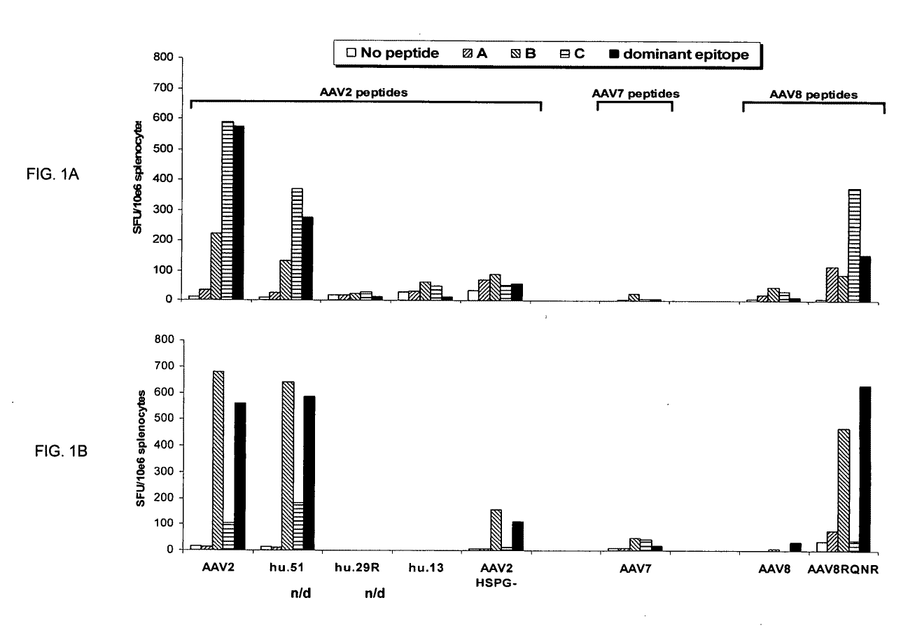

T cell responses are presented in FIG. 1. AAV2 resulted in high T cell frequencies against capsid, however, identical doses of AAV2/7 and 2/8 yielded very little evidence of T cell activation against capsid despite the fact that in vivo transduction was at least five to 10-fold higher with AAV2/7 and 2/8 vectors as compared to AAV2. Serotype specific differences in T cell responses were independent of strain of mice (FIGS. 1A and B) and vector preparation and dose (data not shown).

### Example 2

### Detection of AAV2 Capsid-Specific T Cells in Primate Studies

Similar studies were performed in cynomolgus macaques that received AAV vectors expressing HIV antigens. Cynomolgus macaques were treated and cared for at Barton's West End Facilities (BWEF) at Oxford, N.J.

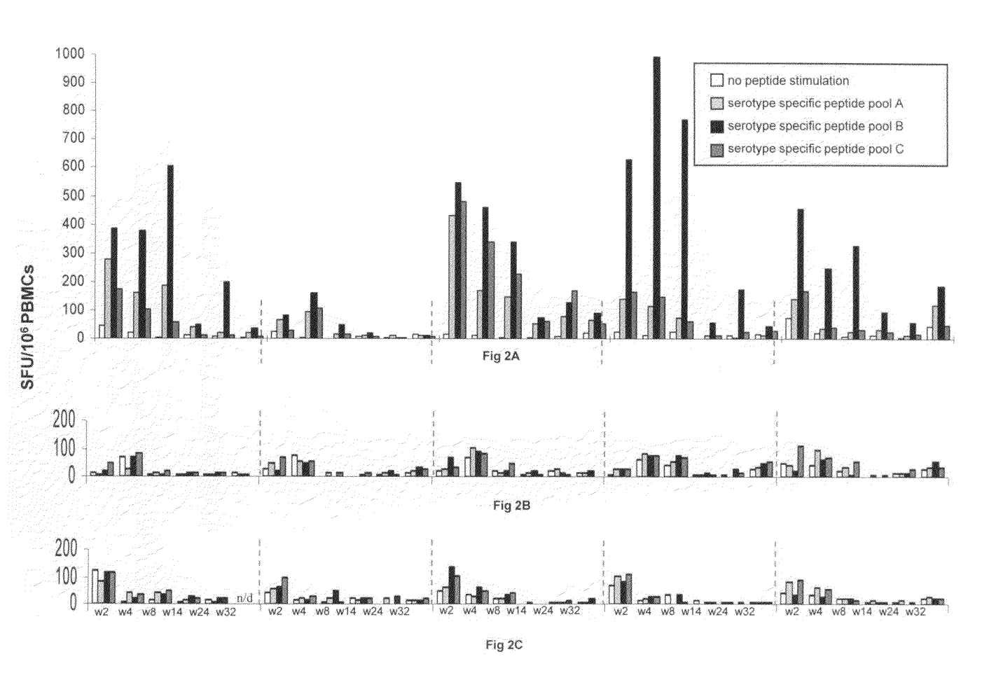

In the primate studies, the following vectors were used AAV.CMV.HIVgp140, AAV.CMV.HIVRT3, and AAV.CMV.HIVGN2. The vectors were packaged with AAV2, 7, or 8 serotypes, as previously described. See, e.g., (See, e.g., FIG. 2, for each serotype three vectors were pooled expressing gp 140, RT and a gag-nef fusion). Animals (5 per group) were injected IM with AAV2, AAV2/7 or AAV2/8. Each mixture of vectors was injected at a dose of 10¹² particles into five animals per group (AAV2, AAV2/7 and AAV2/8). Each monkey was injected intramuscularly at 2 sites at the right quadriceps femoris with a 25-gauge needle with the total mixture of vectors resuspended in 1 ml PBS per animal.

Blood samples were taken via venipuncture of the saphenous vein. Peripheral blood mononuclear cells (PBMCs) were isolated from whole blood and were assayed for capsid specific T cells as previously described [Mueller, Y. M. et al. J Virol 79, 4877-85 (2005)] using pooled VP1 peptides. INF-γ ELISPOT assays were performed using previously described protocols for monkeys [Reyes-Sandoval, A. et al. J Virol 78, 7392-9 (2004)].

Four of the five AAV2 injected animals showed very high T cell frequencies against capsid; most of the AAV2/7 or AAV2/8 dosed animals failed to respond to capsid antigens (i.e., <2.5-fold higher than background). Interestingly, the T cell responses to HIV-1 transgenes in these animals were higher and broader with AAV2/7 and AAV2/8 than with AAV2 indicating an uncoupling of antigen processing and T cell activation for capsids as compared to transgene products (data not shown).

### Example 3

### Identification of T-Cell Epitope in AAV2 Capsid

A. Hybrids Map to AAV T Cell Epitope to the VP3 Protein of AAV2

AAV2/AAV8 hybrids were generated in order to map the domain that directs the activation of T cells

The hybrid capsids between AAV2 and AAV8 used were generated by splicing using overlap extension [Horton, R. M., et al, Gene 77, 61-8 (1989)]. For one pair of hybrids, junctions between AAV2 and AAV8 were engineered at the VP2 start position. Another pair of hybrids was used for which the transition from AAV2 to AAV8 or vice versa is located in a conserved region proximal to the VP3 start codon (660 bp past the VP1 start). These hybrids were used to generate AAV as described.

Analysis of hybrids between AAV2 and AAV8 determined that the domain responsible for directing the T cells to the capsid lies within the VP3 open reading frame. A number of important functional domains are located in VP3 including the previously mapped heparin binding domain characterized by a RXXR motif spanning residues 585 to 588 in AAV2 [Kern, A. et al. J Virol 77, 11072-81 (2003); Opie, S. R., J Virol 77, 6995-7006 (2003).]

To further study the potential role of heparin binding in directing the T cell response to capsid we studied vectors from other members of the Clade B family to which AAV2 belongs, including one that retains the RXXR motif (i.e., hu.51) and two that do not (i.e., hu.29R and hu.13) (Table 1). hu. 29R was optimized for better production after a G396E change. The presence of an intact binding motif correlated with capsid T cell responses (FIG. 1 A and B); hu.13 differs from AAV2 in only two residues other than in the heparin binding domain suggesting this domain is important. Transgene expression of the heparin binding deficient Clade B variants was indistinguishable from that seen with the heparin binding variants in terms of expression of the reporter gene α1 antitrypsin (A1AT) following muscle directed gene transfer (Table 1).

Through these hybrids, the domain was mapped to the RXXR motif on VP3. Evaluation of natural and engineered AAV variants demonstrated direct correlations between heparin binding, uptake into human dendritic cells and activation of capsid T cells. Definitive confirmation of the role of the RXXR motif in directing the capsid T cell response was provided in two engineering experiments.

B. Confirmation of the Role of RXXR Motif in T Cell Activation

The heparin binding site was ablated by converting RGNR to SGNT, which is the consensus sequence from analysis of 15 Clade B, non-heparin binding AAV isolates (Table 1). AAV2HSPG-was generated on the AAV2 packaging plasmid backbone by R585S, R588T mutagenesis, SEQ ID NO: 3, (Quickchange II, Stratagene). The resulting vector did not activate T cells to capsid (FIG. 1A and B).

The corresponding residues in AAV2/8 were converted to a motif that should confer binding to heparin (i.e., QQNT to RQNR [Table 1]). AV8RQNR was site-specifically engineered after Q588R, T591R mutagenesis. The AAV2/8 variant with the reconstructed heparin binding site activated high levels of capsid reactive T cells (FIGS. 1A and B).

For AAV T cell assays, the peptide library for each serotype was divided into three pools such that pool 2A contains the first 50 peptides of AAV2 VP1, pool 2B contains peptides 51-100, and pool 2C contains peptides 101-145. Peptides corresponding to dominant epitopes were obtained from Invitrogen (Carlsbad) or Mimotopes and solubilized in DMSO (4 mg/ml). Dominant H2^{b} restricted epitopes TSNYNKSVN (AAV2), SEQ ID NO: 23, NSLVNPGVA, SEQ ID NO: 24 (AAV7) an NSLANPGIA, SEQ ID NO: 25 (AAV8) were used in the C57Bl/6 mice.

In Table 2, average yield from minimally three vector preparations is given with standard deviation. Gene transfer efficiency in C57Bl/6 (n=5) mice is represented by average and standard deviation of A1AT serum levels following gene delivery with the respective capsid isolate 28 days following intramuscular injection and following intra-portal injection (liver). N/A means not assayed.

TABLE 2

| | Heparin | | Gene transfer efficiency | |
|---|---|---|---|---|
| AAV | column | Vector | (μg/mL) | |
| isolate/mutant | binding | production (GC) | Muscle | Liver |
| AAV2 | + | 1.8 ± 0.8 × 10¹³ | 3.1 ± 0.3 | 4.9 ± 1.5 |
| hu.51 | + | 6.8 ± 3.9 × 10¹² | 2.3 ± 0.5 | 1.9 ± 0.4 |
| AAV8RQNR | + | 1.1 ± 0.2 × 10¹² | n/a | n/a |
| hu.29R | − | 3.7 ± 1.5 × 10¹³ | 2.7 ± 0.5 | 1.8 ± 0.5 |
| hu13 | − | 2.9 ± 1.5 × 10¹³ | 1.8 ± 0.4 | 1.6 ± 0.5 |
| AAV2HSPG- | − | 1.0 ± 0.5 × 10¹³ | n/d | n/d |
| AAV8 | − | 3.2 ± 1.7 × 10¹³ | 38.0 ± 9.3 | 60.1 ± 4.3 |
| AAV7 | n/a | 3.2 ± 2.1 × 10¹³ | 13.4 ± 3.5 | 60.1 ± 12.6 |

The studies described above provide evidence for a direct correlation between the presence of the RxxR heparin binding site and the activation of capsid specific T cells. A subset of these natural and engineered variants was further evaluated for biochemical and cellular evidence of binding to heparin. Purified preparations of vectors were passed over a heparin binding column and the flow through was analyzed for vector genomes. Virtually complete binding of the RXXR containing variants—AAV2, AAV2/hu.51 and AAV2/8RQNR—was observed while substantial quantities of vector were found in the flow through for vectors missing the RXXR motif—AAV2/hu.29R, AAV2/hu.13, AAV2HSPG—and AAV2/8 (Table 2).

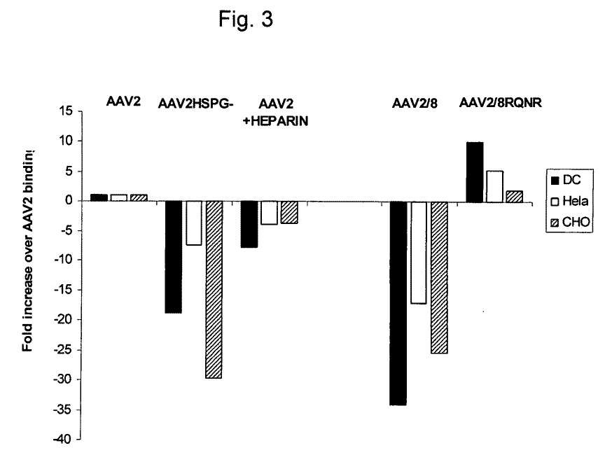

Vectors were also evaluated for binding to HeLa and CHO cells by incubation at 4° C. and analysis of washed cells for retention of vector genomes. Adherent cultures of Hela and CHO cells were maintained according to ATCC and cells were released non-enzymatically after incubation with cell dissociation solution (Sigma-Aldrich). FIG. 3 shows binding relative to that observed with AAV2. Binding of AAV2/8 and the AAV2 variant with the ablated heparin binding site (AAV2HSPG-) is substantially reduced for both cell lines as is binding of AAV2 in the presence of heparin. Reconstruction of the RXXR motif in AAV2/8 confers cell binding to levels in excess to that seen with AAV2.

### Example 4

### Study of Heparin-Mediated Uptake by Dendritic Cells

The emerging hypothesis is that HSP mediated uptake of vector by dendritic cells is a rate limiting step in the activation of T cells against capsid. This was studied in vitro using primary cultures of human monocytye derived dendritic cells.

Human primary dendritic cells were cultured from PBMCs which were provided by the CFAR, University of Pennsylvania. Briefly, plastic adherent monocytes were cultured for 7 days in the presence of GM-CSF (Berlex) and IL-4 (R&D). Immature dendritic cells were phenotyped using the following markers, CD11c, CD80, CD86, CD83, HLA-DR, CD14 and DC-SIGN (BD Biosciences). Viral binding was preceded by 30 min incubation on ice of 10¹⁰ GC in the presence of 20 units of heparin salt (Sigma-Aldrich) or equal volume PBS. Cells (10⁶) were mixed with vector and incubated on a rocking platform at 4° C. After 3 h, cells were recovered by centrifugation and washed three times with serum free culture medium. The cell pellet was suspended in a 400 mM NaCl solution, freeze-thawed three times and the supernatant assayed for the presence of AAV genomes by Taqman PCR.

AAV2 was conjugated with the Alexa Fluor 488 Protein Labeling Kit (Invitrogen). Alexa Fluor 647 Microscale Protein Labeling Kit was used to label the anti-heparan sulfate proteoglycan monoclonal antibody F58-10E4 (Seikagaku, Japan). Cells were incubated at 4° C. for 1 h with virus and antibody in the presence or absence of heparin and subsequently washed three times in a PBS/2.5% FBS/0.1% NaN3. Cells were fixed in a 4% PFA/PBS solution and mixed with an equal volume of Vectashield (Vector Laboratories) before mounting on slide. Microscopy was performed with an inverted Zeiss Axiovert 200M, equipped with Mercury Arc Lamp for epifluoresence, an Apotome unit for z-slices, and blue (DAPI; filterset #49), green (488; filterset #10) or far red (647; filterset #50) filter cubes in place. Images were acquired with a cooled CCD AxioCam HRm camera driven by AxioVision (version 4.3) software. All microscope components (scope, arc lamp, Apotome, filter cubes, camera, software) were obtained from Carl Zeiss MicroImaging.

Binding studies demonstrated identical results to those observed with the cells lines (FIG. 3). All RXXR containing vectors bound dendritic cells while those without this domain did not bind as well.

Binding of AAV to dendritic cells was visualized directly by microscopy using fluorescently labeled AAV2 together with indirect immunofluorescence with an antibody to HSP.

AAV2 bound to the surface of the cells in discrete foci that co-localized with HSP. No detectable binding of AAV2 was observed in the presence of excess heparin.

### Example 5

An immunization study was performed to assess the effect of a variety of AAV having differing capsids on T-cell activation. The study compared a native AAV6 capsid, known to have a heparin binding domain at the lysine residue at position 531 to three modified AAV having capsids with site-specific modifications introduced. These AAV, designed AAV2/6.2 (modified at a position other than K531), AAV2/6.1 (having an AAV6 capsid modified at position 531 to contain a glutamic acid (i.e., a non-conservative amino acid change), and AAV2/6.1.2, having an AAV6 capsid with both the modifications of the AAV6.2 and AAV6.1 capsid were utilized. The sequences and generation of these vectors is described in International Patent Appln No. PCT/US06/13375. AAV1 served as a negative control and AAV2 served as a positive control.

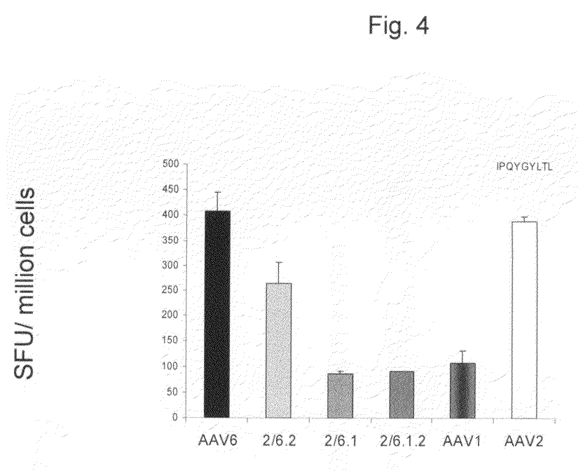

Balb/c mice (male) were immunized intramuscularly with 1×10¹¹ GC AAV2/6, AAV2/6.1, AAV2/6.2, AAV2/6.1.2, AAV2/1 or AAV2 vector. Thirteen (13) days later splenocytes were harvested from 3 mice per group and pooled. Equal amounts of splenocytes were stimulated in vitro with the Balb/c AAV epitope IPQYGYLTL (SEQ ID NO: 1] in a ELISPOT assay. See, FIG. 4.

These results show that viral vector containing an unmodified AAV6 capsid induced levels of T cells comparable to those induced by the AAV2 capsid. In contrast, the modified AAV6 vectors having ablated heparin binding domains (AAV2/6.1 and AAV2/6.1.2) had T-cell responses which are virtually indistinguishable from the negative control (AAV1).

This demonstrates that changing an amino acid residue responsible for mediating heparin binding to an AAV capsid to a non-conservative amino acid residue, not only ablates heparin binding, but also, significantly reduces T cell activation.

### Example 6

### Impact of AAV-Cap Memory (Pre-Existing Immunity) on Heparin Mediated MV Capsid Immunogenicity

In an experiment in which mice immunized with either an Adenoviral vector expressing an irrelevant antigen (SARS nSpike) or the AAV8 VP1 capsid protein simulating a naïve or an AAV pre-immune subject respectively. The immunizing capsid vector is of a serotype different from that of the AAV-administered vector to overcome the neutralizing antibody response induced by the immunization. AAV administration in the presence of antibodies will neutralize the capsid and confound the readout of cellular immune response. In Balb/c mice it has been shown previously [Sabatino, D. E. et al. Mol Ther 12, 1023-33 (2005) and an observation our laboratories, now published as Wang, L., et al, Hum Gene Ther (2007)] to have a conserved MHCI epitope that functionally cross reacts between AAV2 and AAV8. This allows immunization in these mice with one serotype and dose vector of the other. This approach allows memory T-cell responses to be studied in the absence of possible confounding neutralizing antibodies that are not cross-reactive on a distinct AAV serotype.

Several months following immunization these mice were administered either AAV2 or AAV2HSPG—(which has the native AAV2 heparin sulfate binding domain ablated) at different dosages. Seven (7) days following AAV administration, the number of AAV Cap-specific T-cells is measured by a tetramer specific for the dominant AAV Cap epitope by flow cytometry. An expected elevation of AAV capsid T-cells following AAV2 administration, but only minimal T-cell responses to the AAV2HSPG-mutant were observed. In the pre-immune condition, AAV2 administration gave dose responsive elevation of capsid T-cells which was distinctly higher in magnitude to the response in the naive condition. AAV2HSPG-dosed animals at similar and higher doses failed to induce elevated levels of T-cells directed at the capsid.

### Example 7

### Heparin Effect on AAV Genome Biodistribution Following Tail Vein Injection in C57Bl/6 Mice

AAV2, AAV2HSPG-, AAV8 or AAV8RQNR was administered intravenously at a dose of 1×10¹¹ GC. Tissues were harvested and analyzed for presence of vector genomes by quantitative Taqman™ PCR. Tissue distribution was distinct for all vectors and no clear correlates were observed in between non-heparin binding vectors (AAV2HSPG- and AAV8) and the heparin binding ones (AAV2 and AAV8RQNR) with the exception for vector genome presence recovered from spleen.

Heparin binding vector delivered genomes were retrieved at 10-fold higher amounts at the early day 3 time point for all animals that received a heparin binding vector (compared to its non-heparin binding homologue with the exception of one animal that received AAV2 that likely received a partially failed injection due to the lower copy numbers in all tissues of that particular animal). At a day 30 time point following injection, the differences in spleen for AAV2 vs AAV2HSPG-are less clear whereas for AAV8 vs AAV8RQNR, the overall absolute amounts decreased but by 2-logs more for the non-heparin binding AAV8 versus the levels of AAV8RQNR.

The spleen is a secondary lymphoid organ relevant for the activation of T-cells. The finding that heparin binding on AAV2 redirects vector genomes to the spleen is an indication of its higher immunogenicity. Thus, ablation of the heparin binding domain in AAV reduces its immunogenicity.

### Example 8

### Modifications to the Immunogenicity of the Clade A AAV Based vectors while maintaining functionality

A reduced immunogenicity of AAV1 was previously observed in comparison to AAV6 appeared to be correlated with the heparin binding residue on AAV6 (K531 of SEQ ID NO: 4). Even though the immunogenicity of AAV1 is reduced, it is not undetectable by in vivo T cell activation assays.

In a structure function analysis, an additional residue present in both AAV1 and AAV6 was found to be likely responsible for this residual immunogenicity. This positively charged R576 is sterically located in a similar pocket as all residues that was previously identified as implicated in AAV capsid immunogenicity through heparin binding (K531 on AAV6, SEQ ID NO: 4, 585RGNR on AAV2, SEQ ID NO: 3). Only Clade A members (comprising AAV1 and AAV6) carry this R576 residue whereas other serotypes either carry a Glutamic Acid or a Glutamine, a polarity change from positively charged to negatively or uncharged respectively.

AAV6.12 (ablated) vectors have been engineered by site directed mutagenesis with the following changes; either R576Q or R576E of SEQ ID NO: 4. Vectors with these changes produce ˜5-10 times better when compared to AAV6 and equally well as AAV1 or AAV6.1.2. In vivo gene transfer to skeletal muscle in mice is maintained at high levels as measured by hA1AT in the serum following intramuscular administration of AAV encoding CB.hA1AT for the AAV6.1.2R576Q virus. Structural modeling and extrapolation indicate that these R576Q and R576E changes impact on the immunogenicity of the Clade A AAV based vectors while maintaining functionality.

All publications cited in this specification are incorporated herein by reference. While the invention has been described with reference to particularly preferred embodiments, it will be appreciated that modifications can be made without departing from the spirit of the invention.

1. A composition for AAV-mediated delivery of a molecule with reduced AAV immunogenicity, said composition comprising
an adeno-associated virus (AAV) having a modified capsid, wherein said AAV capsid comprises an AAV capsid protein modified to ablate a heparin binding site in the AAV capsid protein;
and a physiologically compatible carrier.

2. The composition according to claim 1, wherein the AAV comprises a capsid protein selected from AAV3, AAV6, hu.51, hu.34, hu.35, hu.45, and hu.47.

3. The composition according to claim 1, wherein the heparin binding site is permanently ablated by site-specific mutagenesis of the sequence encoding the heparin binding site.

4. The composition according to claim 1, wherein the heparin binding site is ablated by binding another specific or a specific molecule to the heparin binding site.

5. The composition according to claim 1, wherein the heparin binding site is blocked by masking the heparin binding site.

6. A composition for AAV-mediated delivery of a molecule with reduced AAV immunogenicity, said composition comprising
a modified AAV having a capsid protein, which capsid protein has been modified to ablate an RxxR (SEQ ID NO: 2) site in the AAV capsid protein and a physiologically compatible carrier.

7. The composition according to claim 6, wherein the AAV has an AAV capsid comprising an AAV vp protein selected from a clade B AAV.

8. The composition according to claim 6, wherein the heparin binding site has a motif characterized by an amino acid sequence RxxR, SEQ ID NO: 2.

9. The composition according to claim 6, wherein the AAV has been modified by altering the first or last arginine in RxxR heparin binding sequence (SEQ ID NO: 2) so that it encodes an amino acid which is non-conservative with arginine.

10. The composition according to claim 9, wherein the heparin binding site is modified at the first amino acid of the RxxR sequence (SEQ ID NO: 2.

11. The composition according to claim 10, wherein the first amino acid of the heparin binding site is changed from Arg to Ser or Glu.

12. The composition according to claim 9, wherein the heparin binding site is modified at the last amino acid of the RxxR sequence.

13. The composition according to claim 12, wherein the last amino acid of the modified heparin binding site is changed from Arg to Thr.

14. (canceled)

15. The composition according to claim 27, wherein said AAV comprises a nucleic acid sequence encoding an immunogenic molecule under the control of sequences which direct expression thereof in a cell.

16. A method of reducing the immunogenicity and/or toxicity of an AAV having a capsid with a heparin binding site, said method comprising the step of
modifying an AAV having a capsid protein with a heparin binding site to ablate heparin binding.

17. The method according to claim 16, further comprising the step of delivering the modified AAV to a subject, whereby the immune response and/or toxicity of the modified AAV is substantially lower than the immune response and/or toxicity of the AAV prior to modifying the AAV to ablate heparin binding.

18. The method according to claim 16, wherein the heparin binding site is ablated by binding another specific or a specific molecule to the heparin binding site.

19. The method according to claim 16, wherein the heparin binding site is blocked by masking the heparin binding site.

20. The method according to claim 16, wherein the heparin binding site is permanently ablated by site-specific mutagenesis of a nucleic acid sequence encoding a heparin binding site.

21. A method of reducing the immunogenicity and/or toxicity of an AAV having a capsid with a RxxR motif, said method comprising the step of
modifying an AAV having a capsid protein with a RxxR motif to replace the first arginine and/or last arginine of this motif with an amino acid which is non-conservative with the arginine.

22. The method according to claim 21, wherein the motif is modified at the first amino acid of the RxxR sequence (SEQ ID NO: 2).

23. The method according to claim 22, wherein the first amino acid of the motif is changed from Arg to Ser or Glu.

24. The method according to claim 22, wherein the motif site is modified at the last amino acid of the RxxR sequence (SEQ ID NO: 2).

25. The method according to claim 22, wherein the last amino acid of the RxxR sequence is changed from Arg to Thr.

26. (canceled)

27. The composition according to claim 1, wherein said AAV comprises a nucleic acid sequence encoding a therapeutic molecule under the control of sequences which direct expression thereof in a cell.

28. A modified AAV prepared according to the method of claim 21.
